(19) United States
(12) Patent Application Publication
(10) Pub. No.: US 2013/0123208 A1
(43) Pub. Date: May 16, 2013

(54) METHOD OF TREATING MULTIPLE SCLEROSIS WITH ADENOSINE RECEPTOR AGONISTS

(71) Applicants: Linda Watkins, Boulder, CO (US); Lisa C. Loram, Cary, NC (US); Jayson M. Rieger, Charlottesville, VA (US)

(72) Inventors: Linda Watkins, Boulder, CO (US); Lisa C. Loram, Cary, NC (US); Jayson M. Rieger, Charlottesville, VA (US)

(73) Assignee: THE REGENTS OF THE UNIVERSITY OF COLORADO, a body corporation, Denver, CO (US)

(21) Appl. No.: 13/697,156

(22) PCT Filed: May 12, 2011
(86) PCT No.: PCT/US11/36270

§ 371 Date: Jan. 28, 2013

Related U.S. Application Data
(60) Provisional application No. 61/334,034, filed on May 12, 2010.

(51) Int. Cl. A61K 31/7076

(52) U.S. Cl. CPC A61K 31/7076
USPC 514/46

(57) ABSTRACT

The present invention includes a composition comprising an A_{2A} agonist. The present invention also includes a method of treating or reducing the symptoms of a neuroinflammatory disease in a patient in need thereof, wherein the method comprises administering to the patient a therapeutically effective amount of an A_{2A} agonist. In one embodiment, the A_{2A} agonist is administered intrathecally to the patient. In another embodiment, the A_{2A} agonist comprises ATL313.

### RELATED APPLICATIONS

This application is related and claims priority to U.S. Provisional Application Ser. No. 61/334,034, filed on May 12, 2010, the contents of which are incorporated herein by reference in their entirety.

### STATEMENT AS TO FEDERALLY FUNDED RESEARCH

This invention was partially funded by NIH Grants DA 024044 & DA017670 from the National Institute of Health. The government may have certain rights in the invention.

### BACKGROUND OF THE INVENTION

Multiple sclerosis (MS) is an autoimmune disease affecting at least 1 in 1000 people in the US and is anticipated to become substantially more prevalent in coming years. This debilitating disease involves an attack by the immune system against antigens of the central nervous system (CNS), especially antigens derived from oligodendrocytes that constitute the myelin sheaths surrounding axons. Initiating factors that cause the disease are poorly understood and are thought to involve a genetic predisposition as well as environmental influences.

In addition to the auto-aggressive T-cells that characterize MS, marked activation of glia (microglia and astrocytes) occurs in MS patients in both the spinal cord and brain. This glial activation results in inflammation involving pro-inflammatory cytokines and chemokines. These inflammatory molecules may profoundly change signaling properties of neurons and lead to demyelination and axonal loss, two hallmarks of MS. Inflammation induced disruption of neuronal signaling in MS patients leads to a diverse array of motor, cognitive, and sensory symptoms. Motor dysfunctions are striking and include spasticity, loss of normal gait, paresis, and progressive ascending paralysis. Decreased sensitivity to touch is a common early sensory symptom that often leads to the initial diagnosis of MS. In addition, neuropathic pain has been documented to occur in a majority of patients. Treatments targeting adaptive immune function appear to be the most effective therapeutic agents to date, but effective pharmacological treatment is still to be elucidated.

Several experimental animal models, including the Experimental Autoimmune Encephalomyelitis (EAE) model, have been developed that produce anatomical and behavioral symptoms that mimic many of those observed in MS. These models have been used to study disease development and progression as well as for pre-clinical testing of potential therapeutics. Many of these models induce autoimmune inflammation by generating an adaptive immune cell mediated attack against antigens contained in the myelin sheaths surrounding axons in CNS. Some of these EAE models, including the one described herein, create a relapsing/remitting course of symptomology, similar to that exhibited in humans.

### SUMMARY OF THE INVENTION

Disclosed herein are therapeutic methods for treating multiple sclerosis, including administering to a patient in need thereof a therapeutically effective amount of an A_{2A} agonist. According to aspects illustrated herein, there is provided a method for treating or reducing the symptoms of multiple sclerosis including intrathecally administering to a patient in need thereof a therapeutically effective amount of an A_{2A} agonist. According to aspects illustrated herein, there is provided a method for treating MS pain including intrathecally administering to a patient in need thereof a therapeutically effective amount of an A_{2A} agonist. According to aspects illustrated herein, there is provided a method for improving motor function including intrathecally administering to a patient in need thereof a therapeutically effective amount of an A_{2A} agonist. According to aspects illustrated herein, there is provided a method for stabilizing motor function including intrathecally administering to a patient in need thereof a therapeutically effective amount of an A_{2A} agonist. According to aspects illustrated herein, there is provided a method for increasing or improving survival rate including intrathecally administering to a patient in need thereof a therapeutically effective amount of an A_{2A} agonist. According to aspects illustrated herein, there is provided a method for increasing remyelination including intrathecally administering to a patient in need thereof a therapeutically effective amount of an A_{2A} agonist. According to aspects illustrated herein, there is provided a method for decreasing or preventing demyelination including intrathecally administering to a patient in need thereof a therapeutically effective amount of an A_{2A} agonist. According to aspects illustrated herein, there is provided a method for sparing neuronal death including intrathecally administering to a patient in need thereof a therapeutically effective amount of an A_{2A} agonist. According to aspects illustrated herein, there is provided a method for suppressing motor paralysis including intrathecally administering to a patient in need thereof a therapeutically effective amount of an A_{2A} agonist.

According to aspects illustrated herein, there is provided a pharmaceutical composition including an A_{2A} agonist in a carrier suitable for intrathecal injection, wherein the concentration of the A_{2A} agonist is from 1-100

According to aspects illustrated herein, there is provided a method for treating or reducing the symptoms of multiple sclerosis including administering to a patient in need thereof a therapeutically effective amount of an A_{2A} agonist.

According to aspects illustrated herein, there is provided a pharmaceutical composition useful for treating multiple sclerosis (e.g., a composition suitable for intrathecal administration), including an effective amount of an A_{2A} agonist and a pharmaceutically acceptable excipient. According to aspects illustrated herein, there is provided a compound described herein for use in medical therapy. According to aspects illustrated herein, there is provided a use of a compound described herein for the manufacture of a medicament for treating multiple sclerosis. These and other aspects of the methods and compositions provided herein relate to the intrathecal administration of an A_{2A} agonist which can attenuate motor symptoms associated with experimental autoimmune encephalitis.

Various embodiments provide certain advantages. Not all embodiments of the invention share the same advantages and those that do may not share them under all circumstances. Further features and advantages of the embodiments, as well as the structures of various embodiments are described in detail below with reference to the accompanying drawings.

### BRIEF DESCRIPTION OF THE FIGURES

FIG. 1 shows an illustrative embodiment of a change in motor symptoms following myelin oligodendrocyte glycoprotein (MOG) intradermal injection into rats;

FIG. 2 shows an illustrative embodiment of a survival curve following A_{2A} agonist administration in rats with EAE;

FIG. 3 shows an illustrative embodiment of an immunohistochemistry of the dorsal spinal cord;

FIGS. 4A and 4B show illustrative embodiments of changes in motor symptoms and a survival curve, respectively, following MOG intradermal injection and intrathecal administration of ATL313 into rats.

FIGS. 5A, 5B and 5C show illustrative sub-analysis of “Responders” of ATL313 in all animals, animals exhibiting full protection and animals exhibiting partial protection.

FIG. 6 illustratively shows that ATL313 markedly enhances survival and arrests paralysis in every surviving ATL313 rat; ˜4-week suppression in other sub-group.

While the above-identified figures set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION OF THE INVENTION

Described herein are therapeutic methods and compositions for the delivery of at least one type of therapeutic agent, such as an adenosine receptor agonist or an anti-inflammatory cytokine, to a patient in need thereof for treatment of a neuroinflammatory disease or a disease associated with demyelination or neuron degeneration. Examples of such diseases include multiple sclerosis (MS), amyotrophic lateral sclerosis, spinal cord injury, spinal stenosis, herniated disks, failed back syndrome, abscess, meningitis, encephalitis, vasculitis, and neuropathic pain involving inflammation of the spinal cord and activation of the glial cells. The methods and compositions herein may also be useful at sites of blood brain barrier disruption. This can be useful for central nervous system (CNS) conditions such as traumatic brain injury, stroke, spinal cord injury, early neuropathic pain, and MS.

Therapeutic agents useful herein include adenosine receptor agonists such as A_{2A} agonists and anti-inflammatory cytokines such as interleukin-10 (IL-10) as well as any other agent which enhances or upregulates the production or release of anti-inflammatory cytokines such as IL-10. Therapeutic agents also include those which suppress the action or release of pro-inflammatory cytokines or are involved with potentiation of opioids for pain control.

In one aspect, the therapeutic methods and compositions increase IL-10 production and/or increase levels of IL-10.

In another aspect, provided herein are compositions including at least one therapeutic agent, such as an adenosine receptor agonist or an anti-inflammatory cytokine, that can be delivered, for example, intrathecally to a patient in need thereof.

In another aspect, the therapeutic methods and compositions provided herein activate the A_{2A} adenosine receptor within the spinal cord for treatment of neuroinflammatory diseases.

Methods and compositions provided herein may be useful for treating a patient suffering from neuroinflammatory diseases, such as MS, and presenting symptoms of neuroinflammatory diseases, such as MS symptoms (herein used interchangeably with signs). They may also useful for symptoms that occur in the progressive stages of the disease such as those seen with the recurrent upsurges of acute disease, classically known as relapses, such as relapsing/remitting disease seen with MS. The methods and compositions herein may reduce the frequency and limit the lasting effects of relapses to relieve symptoms that arise from the release of additional pro-inflammatory cytokines during the relapse, to prevent disability arising from disease progression, and to promote tissue repair. The methods and compositions herein may be useful for increasing the production and/or release of IL-10 in immunocompetent cells of the CNS.

The methods herein may also be useful for treating pain associated with neuroinflammatory diseases, improving or stabilizing motor function, increasing or improving the survival rate, increasing remyelination, reducing neuronal death, suppressing motor paralysis, reducing MRI-detectable brain lesions, preventing or reducing demyelination, or reducing or suppressing spinal cord swelling for patients suffering from neuroinflammatory diseases, such as MS. MRI may be useful for observing edema, demyelination, inflammation, and other anatomical function changes in the brain and spinal cord. Electron microscopy (EM) may be useful for observing the extent of demyelination, remyelination, and neuronal death. Immunohistochemistry may be useful for analyzing spinal glial activation, changes in cell population, the extent of immune cell infiltration, and the phenotype of immune cells

In one aspect, provided herein is a therapeutic method for treating multiple sclerosis, including intrathecally administering to a patient in need thereof a therapeutically effective amount of an A_{2A} agonist. In an embodiment, the A_{2A} agonist is ATL313.

The methods herein may also be useful for inducing an alternatively activated state from the classically activated state in macrophages or microglia to treat neuroinflammatory diseases, such as MS. The methods may cause alteration in morphology, activation and/or phenotype of recruited or resident monocyte derived cells (macrophages and microglia) in the spinal cord. The methods may be useful for phenotypically switching glia and macrophages from proinflammatory state to an anti-inflammatory state, which can be useful, for example, in controlling demyelination process and other MS symptoms. Markers of alternatively activated macrophages include but are not limited to upregulation of arginase-1 (Arg-1), Ym1, and Fizz1. These markers can be analyzed using biochemical techniques such as PCR and western blot analyses.

In an embodiment, the number of injections administered to the patient is a single injection, such as in a bolus. In an embodiment, repeated injections are administered to the patient. In an embodiment, the number of repeated injections administered to the patient is at least two or at least three injections.

The therapeutic method described herein can be administered to a patient in a daily, weekly (e.g., 1, 2, 3, 4, 5, 6, or 8 weeks between administrations), biweekly, monthly, bimonthly, quarterly, or yearly regimen. The therapeutic method may be administered using a combination of time periods. The therapeutic method may be administered based on individual or group response rates that are predetermined prior to beginning the method, predetermined during the method or are customized based on individual results at the time of or within a few hours or days of the next administration. For example, one administration may be given at four weeks and another may be given two months following the previous administration. Follow-up administrations of the therapeutic method can also be given at a time point where symptoms just begin to worsen from the maximal symptom relief attained by the previous administration. The therapeutic agent used may also be different or the same for the various administrations.

Symptoms of MS may vary with timing, type, and severity and may include motor, cognitive, and sensory symptoms. Symptoms of motor dysfunction may include spasticity, loss of normal gait, paresis, and progressive ascending paralysis. An early sensory symptom may be decreased sensitivity to touch as well as reduced pain sensitivity. Cognitive deficits may include learning and memory dysfunction such as difficulties with concentration, attention, memory, and poor judgment. Symptoms of neuropathic pain such as allodynia (perception of normally non-painful stimuli as painful) and thermal and mechanical hyperalgesia (exaggerated pain sensitivity) may also be experienced by MS patients. MS symptoms can go through relapses and periods of remission. Some MS patients experience pain, which can be measured using different methodologies (see e.g. Pain 137 (2008), 96-111).

Other non-limiting examples of MS symptoms may include numbness, tingling, pringling, “pins and needles” sensations, muscle weakness in extremities, muscle spasms, tremors, spasticity, cramps, pain, blindness, blurred or double vision, red-green color distortion, incontinence, urinary urgency or hesitancy, constipation, speech impediments, loss of sexual function, nausea, disabling fatigue, depression, short term memory problems, hearing loss, other forms of cognitive dysfunction, inability to swallow, inability to control breathing, difficulty with coordination and balance, dizziness, difficulty walking or standing, or partial or complete paralysis.

In an embodiment, at least one symptom of multiple sclerosis remains partially, transiently, substantially, or completely eliminated for less than an hour, at least about 4 hours, 6 hours, 12 hours, 1 day, 1 week, 4 weeks, 1 month, 2 months, 3 months, 4 months, 5 months, 6 months, 9 months, one year, 18 months, two years, three years, four years, five years, or more than five years after the termination of the therapeutic method, or permanently after the termination of the therapeutic method. In an embodiment, provided herein is a method of alleviating symptoms of multiple sclerosis by administering to a patient a therapeutically effective amount of an A_{2A} agonist wherein symptoms of MS are substantially eliminated for at least about one month after treatment is terminated.

In an embodiment, at least one or more symptoms of MS are alleviated during periods of relapse by administering to a patient in need thereof a therapeutically effective amount of an A_{2A} agonist such as ATL313. In an embodiment, at least one or more symptoms of MS are stabilized during periods of relapse by administering to a patient in need thereof a therapeutically effective amount of an A_{2A} agonist. In an embodiment, administration of a therapeutically effective amount of an A_{2A} agonist to a patient in need thereof results in an extended period of remission of at least one MS symptom when compared to the period of remission of the patient prior to administration of the an A_{2A} agonist. In an embodiment, the period of remission is increased by at least: 1, 2, 3, 4, 5, 10, 15, 20, 25, 30, 60, 80, or 100 days.

Any methodology, standard, or scale used to measure symptoms of neuroinflammatory diseases (e.g. MS) can be used herein. Symptoms are measured using a variety of tools such as those methods of measuring pain reviewed in Pain 137 (2008) 96-111. Also, any future methodology, standard, or scale used for measuring MS pain or symptoms can be used herein.

In an embodiment, a therapeutic composition provided herein is administered to a patient in need thereof (e.g. a patient having a neuroinflammatory disease, such as MS) and increases the survival rate of the patient. In an embodiment, the survival rate may be significantly increased in comparison with no treatment, a standard of care, another treatment or other comparable as would be understood in the art. In an embodiment, the survival rate is increased by at least about: 5%, 10%, 25%, 50%, 75%, 100%, 110%, 120%, 130%, 140%, 150%, 160%, 170%, 180%, 190%, 200%, 250%, 300%, 350%, 400%, 500%, 750%, 1000% or more. The survival rate may be measured using any known methodology or standard.

In an embodiment, the volume administered is in the range of about: 20, 40, 60, 80, 100, 120, 140, 160, 180, 200, 220, 240, 260, 280, 300, 320, 340, 360, 380, 400, 420, 440, 460, 480, 500, 520, 540, 560, 580, 600, 650, 700, 750, 800, 850, 900, 950, to 1000 μL. In an embodiment, the volume administered is about: 20 to 500 μL, 500 to 1000 μL, 100 to 200 μL, 100 to 150 μL, or 150 to 200 μL. In an embodiment, the volume administered is at least about: 20, 40, 60, 80, 100, 120, 140, 160, 180, 200, 220, 240, 260, 280, 300, 320, 340, 360, 380, 400, 420, 440, 460, 480, 500, 520, 540, 560, 580, 600, 650, 700, 750, 800, 850, 900, 950, or 1000 μL. In an embodiment, the volume administered is about 100 to 200 μL.

In an embodiment, the amount of A_{2A} agonist administered is from about: 0.1, 0.2, 0.3, 0.4, 0.5, 0.6, 0.7, 0.8, 0.9, 1, 2, 3, 4, 5, 6, 7, 8, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, to 50 ng/kg per administration. In an embodiment, the amount of A_{2A} agonist administered is about: 0.1 to 25 ng/kg, 50 to 100 ng/kg, 0.5 to 15 ng/kg, 0.5 to 10 ng/kg, or 10 to 15 ng/kg. In an embodiment, the amount of A_{2A} agonist administered is at least about: 0.1, 0.2, 0.3, 0.4, 0.5, 0.6, 0.7, 0.8, 0.9, 1, 2, 3, 4, 5, 6, 7, 8, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 ng/kg per administration.

In an embodiment, provided herein is a pharmaceutical composition including: an A_{2A} agonist in a carrier suitable for intrathecal injection and the concentration of the A_{2A} agonist is from about: 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 30, 40, 50, 60, 70, 80, 90, to 100 μM. In an embodiment, the concentration of the A_{2A} agonist is about: 0.1 to 1 μM, 0.1 to 20 μM, 0.1 to 50 μM, 1 to 50 μM, 1 to 20 μM, 5 to 10 μM, 10 to 20 μM, 20 to 50 μM or 50 to 100 μM. In an embodiment, the concentration of the A_{2A} agonist is at least about: 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 30, 40, 50, 60, 70, 80, 90, or 100 μM.

In an embodiment, a second volume is administered to facilitate delivery of the therapeutic agent, wherein the second volume includes an intrathecally suitable carrier. In an embodiment, the second volume includes an intrathecally suitable carrier that is the same as that in the pharmaceutical composition including the therapeutic agent. In an embodiment, the second volume includes an intrathecally suitable carrier different from that in the pharmaceutical composition including the therapeutic agent. In an embodiment, the second volume administered is in the range from about: 0.5, 1, 1.5, 2, 2.5, 3, 3.5, 4, 4.5, to 5 mL. In an embodiment, the second volume administered is in the range of at least about: 0.5, 1, 1.5, 2, 2.5, 3, 3.5, 4, 4.5, or 5 mL. In an embodiment, the second volume administered is at least about: 20, 40, 60, 80, 100, 120, 140, 160, 180, 200, 220, 240, 260, 280, 300, 320, 340, 360, 380, 400, 420, 440, 460, 480, 500, 520, 540, 560, 580, 600, 650, 700, 750, 800, 850, 900, 950, to 1000 μL.

In an embodiment, a pharmaceutical composition includes at least one A_{2A} agonist. In an embodiment, the pharmaceutical composition includes more than one type of A_{2A} agonist. In an embodiment, the pharmaceutical composition includes at least one A_{2A} agonist and at least one other active agent for the treatment of neuroinflammatory diseases. Any A_{2A} agonist that is not disclosed herein but which may be useful for the amelioration or treatment of neuroinflammatory diseases are included and intended within the scope of the embodiments presented. In an embodiment, pharmaceutically active metabolites, salts, polymorphs, prodrugs, analogues, and/or derivatives of the A_{2A} agonist disclosed herein that retain the ability of the parent A_{2A} agonist to treat neuroinflammatory diseases are useful in the formulations.

The therapeutic method or compositions provided herein are administered to the patient at any time during the disease progression. In an embodiment, the therapeutic method or composition is administered to the patient late in disease progression. In an embodiment, the therapeutic method or composition is administered to the patient during the early or middle stages of the disease. In an embodiment, the therapeutic method or composition is administered to the patient during a period of disease relapse. In an embodiment, the therapeutic method or composition is administered to the patient during a period of disease remission whereby a future period of relapse is prevented or delayed.

The therapeutic methods and compositions provided herein may be administered in combination with other methods of treatment for neuroinflammatory diseases. In an embodiment, the neuroinflammatory disease is MS. The therapeutic methods herein can be administered prior to, simultaneously with, or following other methods of MS treatment. Non-limiting examples of other methods of treatment include physical therapy or exercise, beta interferon (or interferon beta), co-polymer I or glatiramer acetate, mitoxantrone, azathioprine, natalizumab, steroids, and/or muscle relaxants and tranquilizers such as baclofen, tizanidine, diazepam, clonazepam, and/or dantrolene.

In an embodiment, the A_{2A} agonists treat MS symptoms by enhancing IL-10 production and/or release. In an embodiment, the A_{2A} agonists treat MS symptoms by enhancing both IL-10 and IL-4 production and/or release. In an embodiment, the A_{2A} agonists reduce or prevent hippocampal neuropathology and dysfunction in MS.

Examples of A_{2A} agonists that are useful in the compositions and practice of the methods are described herein. Embodiments listed herein for radicals, substituents, and ranges, are for illustration only; they do not exclude other defined values or other values within defined ranges for the radicals and substituents.

Examples of A_{2A} agonists include A_{2A} agonists described in: U.S. Pat. No. 6,232,297 to Linden, et al. which describes compounds having the general formula:

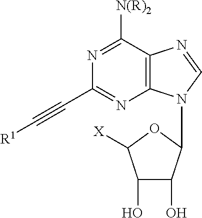

wherein each R can be H, X can be ethylaminocarbonyl and R¹ is 4-carboxycyclohexylmethyl (DWH-146a), R¹ is 4-methoxycarbonylcyclohexylmethyl (DWH-146e) or R¹ is 4-acetoxymethyl-cyclohexylmethyl (JMR-193);

U.S. Pat. No. 7,214,665 to Linden, et al. which describes compounds having the general formula:

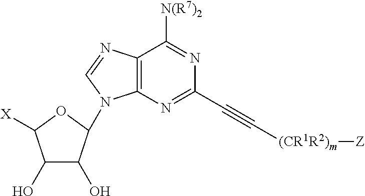

wherein R⁷ can be H, X can be an ether or an amide, CR¹R² can be CH₂, and Z can be a heterocyclic ring;

U.S. Patent Application No. 2006/0040889 to Rieger, et al. which describes compounds having the general formula:

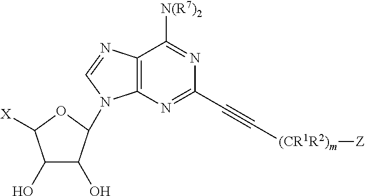

wherein R⁷ can be H, X can be a cycloalkyl-substituted ether or amide, CR¹R² can be CH₂, and Z can be a heterocyclic ring;

U.S. Patent Application No. 2007/0270373 to Rieger, et al. which describes compounds having the general formula:

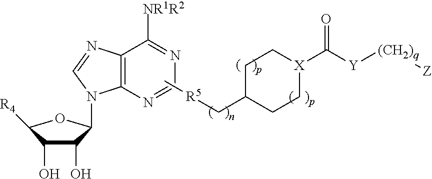

wherein NR¹R² can be NH₂, R⁴ can be a an ether or an amide, R⁵ can be ethynyl, Y can be O or NR¹, and Z can be an aryl or heteroaryl;

U.S. Patent Application Nos. 2009/0162282 and 2009/0162292 to Thompson, et al and Rieger, et al., respectively, which describe compounds having the general formulae:

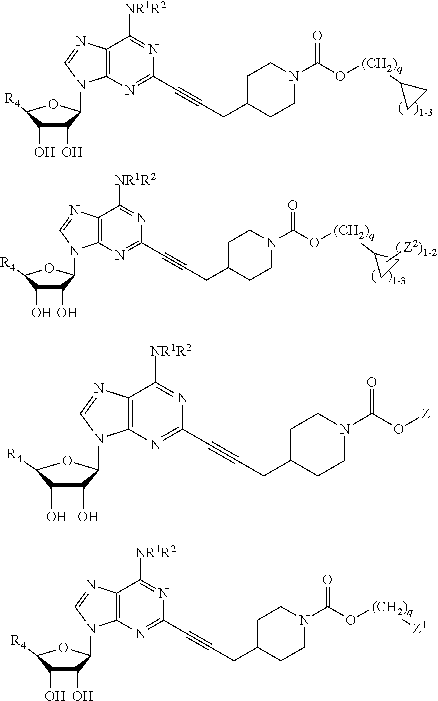

wherein NR¹R² can be NH₂, R⁴ can be an ether or an amide, and Z can be a ring;

U.S. Pat. No. 5,593,975 and to Cristall: which describes 2-arylethynyl, 2-cycloalkylethynyl or 2-hydroxyalkylethynyl derivatives, wherein the riboside residue is substituted by carboxy amino, or substituted carboxy amino; all of which are reported to be A_{2A} agonists.

In an embodiment, an A_{2A} agonist is a compound of formula I:

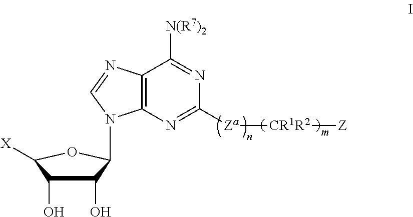

wherein

Z^{a} is C≡C, O, NH, or NHN═CR^{3a};

n is 0 or 1;

each R¹ is independently hydrogen, halo, —OR^{a}, —SR^{a}, (C₁-C₈)alkyl, cyano, nitro, trifluoromethyl, trifluoromethoxy, (C₃-C₈)cycloalkyl, heterocycle, heterocycle(C₁-C₈)alkylene-, aryl, aryl(C₁-C₈)alkylene-, heteroaryl, heteroaryl(C₁-C₈)alkylene-, —CO₂R^{a}, R^{a}C(═O)O—, R^{a}C(═O)—, —OCO₂R^{a}, R^{b}R^{c}NC(═O)O—,

R^{a}OC(═O)N(R^{b})—, R^{b}R^{c}N—, R^{b}R^{c}NC(═O)—, R^{a}C(═O)N(R^{b})—, R^{b}R^{c}NC(═O)N(R^{b})—, R^{b}R^{c}NC(═S)N(R^{b})—, —OPO₃R^{a}, R^{a}OC(═S)—, R^{a}C(═S)—, —SSR^{a}, R^{a}S(═O)—, R^{d}S(═O)₂—, or —N═NR^{b};

each R² is independently hydrogen, halo, (C₁-C₈)alkyl, (C₃-C₈)cycloalkyl, heterocycle, heterocycle(C₁-C₈)alkylene-, aryl, aryl(C₁-C₈)alkylene-, heteroaryl, or heteroaryl(C₁-C₈)alkylene-;

alternatively, R¹ and R² and the carbon atom to which they are attached is C═O, C═S or C═NR^{d},

Z is CR³R⁴R⁵ or NR⁴R⁵;

R⁴ and R⁵ are independently H or (C₁-C₈)alkyl wherein R⁴ and R⁵ are independently substituted with 0 to 3 R⁶ groups; or

alternatively, R⁴ and R⁵ together with the atom to which they are attached form a saturated, partially unsaturated, or aromatic ring that is mono-, bi- or polycyclic and has 3, 4, 5, 6, 7, 8, 9 or 10 ring atoms optionally having 1, 2, 3, or 4 heteroatoms selected from —O—, S(O)₀₋₂, and amine in the ring;

wherein the ring including R⁴ and R⁵ is unsubstituted or substituted with from 1 to 6 R⁶ groups;

wherein each R⁶ is independently hydrogen, halo, —OR^{a}, —SR^{a}, (C₁-C₈)alkyl, cyano, nitro, trifluoromethyl, trifluoromethoxy, (C₁-C₈)cycloalkyl, (C₆-C₁₂)bicycloalkyl, heterocycle, heterocycle (C₁-C₈)alkylene-, aryl, aryl (C₁-C₈)alkylene-, heteroaryl, heteroaryl(C₁-C₈)alkylene-, —CO₂R^{a}, R^{a}C(═O)O—, R^{a}C(═O)—, —OCO₂R^{a}, R^{b}R^{c}NC(═O)O—, R^{a}OC(═O)N(R^{b})—, R^{b}R^{c}N—, R^{b}R^{c}NC(═O)—, R^{a}C(═O)N(R^{b})—, R^{b}R^{c}NC(═O)N(R^{b})—, R^{b}R^{c}NC(═S)N(R^{b})—, —OPO₃R^{a}, R^{a}OC(═S)—, R^{a}C(═S)—, —SSR^{a}, R^{a}S(═O)—, —NNR^{b}, or two R⁶ groups and the atom to which they are attached is C═O, C═S; or two R⁶ groups together with the atom or atoms to which they are attached can form a carbocyclic or heterocyclic ring including from 1 to 6 carbon atoms and optionally 1, 2, 3, or 4 heteroatoms selected from —O—, S(O)₀₋₂, and amine in the ring;

R³ is hydrogen, halo, —OR^{a}, —SR^{a}, (C₁-C₈)alkyl, cyano, nitro, trifluoromethyl, trifluoromethoxy, (C₃-C₈)cycloalkyl, heterocycle, heterocycle(C₁-C₈)alkylene-, aryl, aryl(C₁-C₈)alkylene-, heteroaryl, heteroaryl(C₁-C₈)alkylene-, —CO₂R^{a}, R^{a}C(═O)O—, R^{a}C(═O)—, —OCO₂R^{a}, R^{b}R^{c}NC(═O)O—, R^{a}OC(═O)N(R^{b})—, R^{b}R^{c}N—, R^{b}R^{c}NC(═O)—, R^{a}C(═O)N(R^{b})—, R^{b}R^{c}NC(═O)N(R^{b})—, R^{b}R^{c}NC(═S)N(R^{b})—, —OPO₃R^{a}, R^{a}OC(═S)—, R^{a}C(═S)—, —SSR^{a}, R^{a}S(═O)—, R^{a}S(═O)₂—, —NNR^{b}; or if the ring formed from CR⁴R⁵ is aryl or heteroaryl or partially unsaturated then R³ can be absent;

R^{3a} is hydrogen, (C₁-C₈)alkyl, or aryl;

each instance of R⁷ is independently hydrogen, (C₁-C₈)alkyl, (C₃-C₈)cycloalkyl, aryl, aryl(C₁-C₈)alkylene, heteroaryl, or heteroaryl(C₁-C₈)alkylene-;

X is —CH₂OR^{a}, —CO₂R^{a}, —CH₂OC(O)R^{a}, —C(O)NR^{b}R^{c}, —CH₂SR^{a}, —C(S)OR^{a}, —CH₂OC(S)R^{a}, —C(S)NR^{b}R^{c}, or —CH₂N(R^{b})(R^{c});

alternatively, X is a group having the formula:

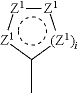

wherein each Z¹ is independently —O—, S(O)₀₋₂, —C(R⁸)—, or —NR⁸—, provided that at least one Z¹ is —O—, S(O)₀₋₂, or —NR⁸—;

each R⁸ is independently hydrogen, (C₁-C₈)alkyl, (C₁-C₈)alkenyl, (C₃-C₃)cycloalkyl, (C₃-C₈)cycloalkyl(C₁-C₈)alkylene, (C₃-C₈)cycloalkenyl, (C₃-C₈)cycloalkenyl(C₁-C₈)alkylene, aryl, aryl(C₁-C₈)alkylene, heteroaryl, or heteroaryl(C₁-C₈)alkylene, wherein any of the alkyl or alkenyl groups of R⁸ are optionally interrupted by —O—, —S—, or —N(R^{a})—;

wherein any of the alkyl, cycloalkyl, heterocycle, aryl, or heteroaryl, groups of R¹, R², R³, R^{3a}, R⁶, R⁷ and R⁸ is optionally substituted on carbon with one or more (e.g. 1, 2, 3, or 4) substituents selected from the group consisting of halo, —OR^{a}, —SR^{a}, (C₁-C₈)alkyl, cyano, nitro, trifluoromethyl, trifluoromethoxy, (C₃-C₈)cycloalkyl, (C₆-C₁₂)bicycloalkyl, heterocycle, heterocycle(C₁-C₈)alkylene-, aryl, aryloxy, aryl(C₁-C₈)alkylene-, heteroaryl, heteroaryl(C₁-C₈)alkylene-, —CO₂R^{a}, R^{a}C(═O)O—, R^{a}C(═O)—, —OCO₂R^{a}, R^{b}R^{c}NC(═O)O—, R^{a}OC(═O)N(R^{b})—, R^{b}R^{c}NC(═O)—, R^{a}C(═O)N(R^{b})—, R^{b}R^{c}NC(═O)N(R^{b})—, R^{b}R^{c}NC(═S)N(R^{b})—, —OPO₃R^{a}, R^{a}OC(═S)—, R^{a}C(═S)—, —SSR^{a}, R^{a}S(═O)ₚ—, R^{b}R^{c}NS(O)ₚ—, and —N═NR^{b};

wherein any (C₁-C₈)alkyl, (C₃-C₈)cycloalkyl, (C₆-C₁₂)bicycloalkyl, (C₁-C₈)alkoxy, (C₁-C₈)alkanoyl, (C₁-C₈)alkylene, or heterocycle, is optionally partially unsaturated;

each R^{a}, R^{b} and R^{c} is independently hydrogen, (C₁-C₁₂)alkyl, (C₁-C₈)alkoxy, (C₁-C₈)alkoxy-(C₁-C₁₂)alkylene, (C₃-C₈)cycloalkyl, (C₃-C₈)cycloalkyl-(C₁-C₁₂)alkylene, (C₁-C₈)alkylthio, amino acid, aryl, aryl(C₁-C₈)alkylene, heterocycle, heterocycle-(C₁-C₈)alkylene, heteroaryl, or heteroaryl(C₁-C₈)alkylene; or

alternatively R^{b} and R^{c}, together with the nitrogen to which they are attached, form a pyrrolidino, piperidino, morpholino, or thiomorpholino ring;

wherein any of the alkyl, cycloalkyl, heterocycle, aryl, or heteroaryl groups of R^{a}, R^{b} and R^{c} is optionally substituted on carbon with 1 or 2 substituents selected from the group consisting of halo, —(CH₂)ₐOR^{e}, —(CH₂)ₐSR^{e}, (C₁-C₈)alkyl, (CH₂)ₐCN, (CH₂)ₐNO₂, trifluoromethyl, trifluoromethoxy, —(CH₂)ₐCO₂R³, (CH₂)ₐNR^{e}R^{e}, and (CH₂)ₐC(O)NR^{e}R^{e};

R^{d} is hydrogen or (C₁-C₆)alkyl;

R^{e} is independently selected from H and (C₁-C₆)alkyl;

a is 0, 1, or 2;

i is 1 or 2

m is 0 to 8; and

p is 0 to 2;

provided that m is at least 1 when Z is NR⁴R⁵; or

a pharmaceutically acceptable salt thereof.

In an embodiment, the A_{2A} agonist is a compound of formula I wherein Z^{a} is C≡C and n is 1.

In an embodiment, the A_{2A} agonist is a compound of formula (Ia):

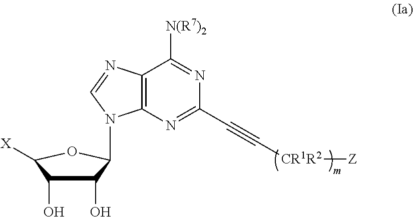

wherein

R¹ is each independently hydrogen, —OH, —CH₂OH, —OMe, —OAc, —NH₂, —NHMe, —NMe₂ or —NHAc;

R² is each independently hydrogen, (C₁-C₈)alkyl, cyclopropyl, cyclohexyl or benzyl;

Z is CR³R⁴R⁵ or NR⁴R⁵ and R⁴ and R⁵, together with the atom to which they are attached, are linked to form a ring a 3-10 membered carbocyclic, heterocyclic or aromatic ring wherein said ring is unsubstituted or optionally substituted with one or more of R⁶;

R³ is hydrogen, OH, OMe, OAc, NH₂, NHMe, NMe₂ or NHAc;

R⁶ is each independently hydrogen, (C₁-C₈)alkyl, —OR^{a}, —CO₂R^{a}, R^{a}C(═O)—, R^{a}C(═O)O—, R^{b}R^{c}N—, R^{b}R^{c}NC(═O)—, or aryl;

R^{a}, R^{b} and R^{c} are each independently hydrogen, (C₃-C₄)cycloalkyl, (C₁-C₈)alkyl, aryl or aryl(C₁-C₈)alkylene;

each R⁷ is independently hydrogen, alkyl (e.g., C₁-C₈alkyl), aryl, aryl(C₁-C₈)alkylene or heteroaryl(C₁-C₈)alkylene;

X is —CH₂OR^{a}, —CO₂R^{a}, —CH₂OC(O)R^{a}, or —C(O)NR^{b}R^{c} or selected from the group consisting of:

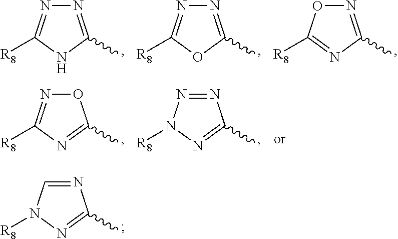

R⁸ is methyl, ethyl, propyl, 2-propenyl, cyclopropyl, cyclobutyl, cyclopropylmethyl, —(CH₂)₂CO₂CH₃, or —(CH₂)₂₋₃OH; and

m is 0, 1 or 2;

or a pharmaceutically acceptable salt thereof.

In an embodiment, the 3-10 membered carbocyclic, heterocyclic or aromatic ring is selected from the group consisting of cyclopentane, cyclohexane, piperidine, dihydro-pyridine, tetrahydro-pyridine, pyridine, piperazine, tetrahydro-pyrazine, dihydro-pyrazine, pyrazine, dihydro-pyrimidine, tetrahydro-pyrimidine, hexahydro-pyrimidine, pyrazine, imidazole, dihydro-imidazole, imidazolidine, pyrazole, dihydro-pyrazole, or pyrazolidine; wherein the 3-10 membered carbocyclic, heterocyclic or aromatic ring is unsubstituted or substituted with R⁶ groups.

In a an embodiment, the 3-10 membered carbocyclic, heterocyclic or aromatic ring is selected from the group consisting of:

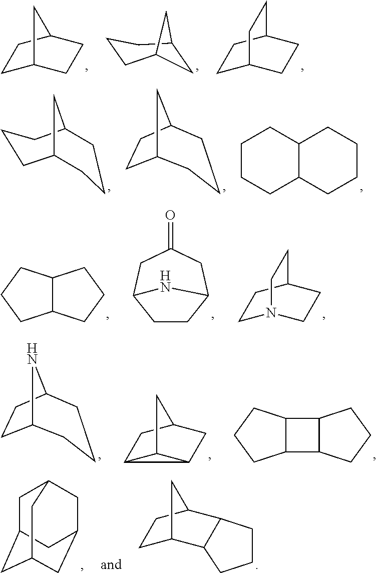

In an embodiment, the A_{2A} agonist is a compound of formula (Ia) wherein:

R¹ is each independently hydrogen, OH, OMe, or NH₂;

R² is each independently hydrogen, methyl, ethyl or propyl;

Z is CR³R⁴R⁵ or NR⁴R⁵ and R⁴ and R⁵, together with the atom to which they are attached, are linked to form a ring a 3-10 membered carbocyclic, heterocyclic or aromatic ring wherein said ring is unsubstituted or optionally substituted with one or more of R⁶;

R³ is hydrogen, OH, OMe, or NH₂;

R⁶ is each independently hydrogen, (C₁-C₈)alkyl, —OR^{a}, —CO₂R^{a}, R^{a}C(═O)—, R^{a}C(═O)O—, R^{b}R^{c}NC(═O)—, or aryl;

R^{a}, R^{b} and R^{c} are independently hydrogen, methyl, ethyl, propyl, butyl, ethylhexyl, cyclopropyl, cyclobutyl, phenyl or benzyl;

N(R⁷)₂ is amino, methylamino, dimethylamino; ethylamino; pentylamino, diphenylethylamino, (pyridinylmethyl)amino, (pyridinyl)(methyl)amino, diethylamino or benzylamino; and,

X is —CH₂OR^{a} or —C(O)NR^{b}R^{c} or selected from the group consisting of:

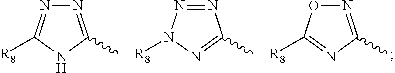

R⁸ is methyl, ethyl, propyl, or cyclopropyl; and

m is 0, 1 or 2;

or a pharmaceutically acceptable salt thereof.

In an embodiment, Z is selected from the group consisting of:

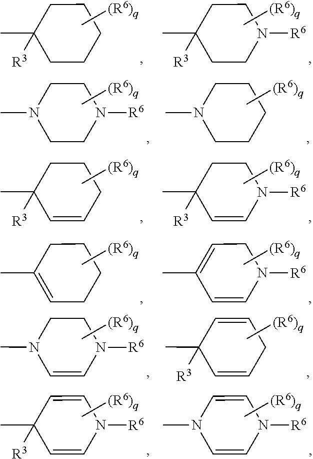

wherein q is from 0 to 4 (e.g., 0-2);

R³ is hydrogen, OH, OMe, or NH₂; and

R⁶ is each independently hydrogen, (C₁-C₈)alkyl, —OR^{a}, —CO₂R^{a}, R^{a}C(═O)—, R^{a}C(═O)O—, R^{b}R^{c}N—, R^{b}R^{c}NC(═O)—, or aryl.

In a an embodiment, the 3-10 membered carbocyclic, heterocyclic or aromatic ring is selected from the group consisting of:

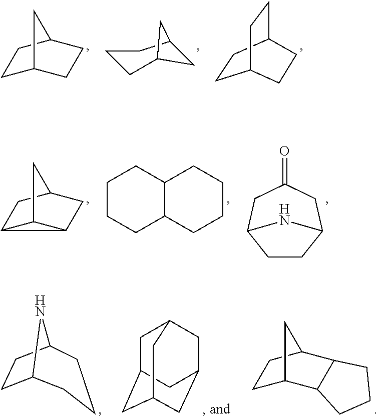

In an embodiment, the A_{2A} agonist is a compound of formula (Ia) wherein:

R¹ is each independently hydrogen, OH, or NH₂;

R² is each independently hydrogen or methyl;

Z is selected from the group consisting of:

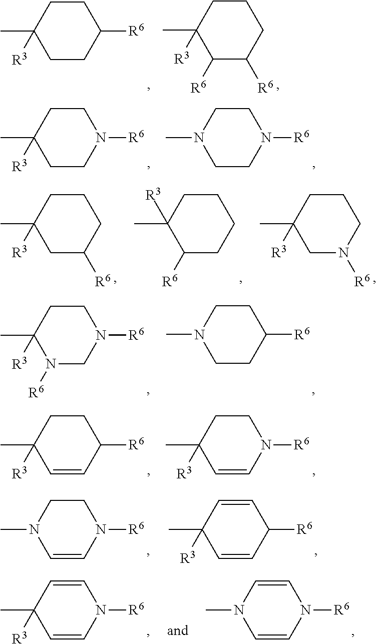

wherein

q is from 0 to 2;

R³ is hydrogen, OH, or NH₂;

R⁶ is hydrogen, methyl, ethyl, t-butyl, phenyl, —CO₂R^{a}—CONR^{b}R^{c}, or R^{a}C(═O)—;

R^{b} is H;

R^{a} is methyl, ethyl, propyl, butyl, pentyl, ethylhexyl cyclopropyl, and cyclobutyl; —N(R⁷)₂ is amino, methylamino, dimethylamino; ethylamino; diethylamino or benzylamino;

or a pharmaceutically acceptable salt thereof.

In an embodiment, Z is CR³R⁴R⁵ or NR⁴R⁵ and R⁴ and R⁵, together with the atom to which they are attached, are linked to form a ring a 3-10 membered carbocyclic, heterocyclic or aromatic ring wherein the 3-10 membered carbocyclic, heterocyclic or aromatic ring is selected from the group consisting of:

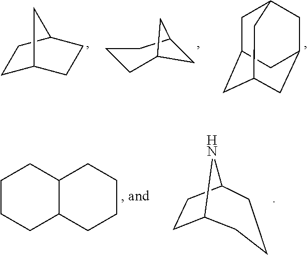

In an embodiment, the A_{2A} agonist is a compound of formula (Ia) wherein:

R¹ is each independently hydrogen or OH;

R² is hydrogen;

Z is selected from the group consisting of:

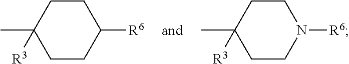

R³ is hydrogen or OH;

R⁶ is hydrogen, methyl, ethyl, —CO₂R^{a}, or —CONR^{b}R^{c};

R^{a} is methyl, ethyl, isopropyl, isobutyl, tert-butyl, or cyclopropyl;

R^{b} and R^{c} is H;

N(R⁷)₂ is amino or methylamino;

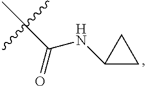 [SMILES: *C(=O)NC1CC1]

X is —CH₂OH, C(O)NHCH₃, or —C(O)NHCH₂CH₃;

or a pharmaceutically acceptable salt thereof.

In an embodiment, Z is 2-methyl cyclohexane, 2,2-dimethylcyclohexane, 2-phenylcyclohexane, 2-ethylcyclohexane, 2,2-diethylcyclohexane, 2-tert-butyl cyclohexane, 3-methyl cyclohexane, 3,3-dimethylcyclohexane, 4-methyl cyclohexane, 4-ethylcyclohexane, 4-phenyl cyclohexane, 4-tert-butyl cyclohexane, 4-carboxymethyl cyclohexane, 4-carboxyethyl cyclohexane, 3,3,5,5-tetramethyl cyclohexane, 2,4-dimethyl cyclopentane, 4-cyclohexanecarboxylic acid, 4-cyclohexanecarboxylic acid esters, 4-methyloxyalkanoyl-cyclohexane, 4-piperidine-1-carboxylic acid methyl ester, 4-piperidine-1-carboxylic acid tert-butyl ester 4-piperidine, 4-piperazine-1-carboxylic acid methyl ester, 4-piperidine-1-carboxylic acid tert-butylester, 1-piperidine-4-carboxylic acid methyl ester, 1-piperidine-4-carboxylic acid tert-butyl ester, tert-butylester, 1-piperidine-4-carboxylic acid methyl ester, or 1-piperidine-4-carboxylic acid tert-butyl ester, 3-piperidine-1-carboxylic acid methyl ester, 3-piperidine-1-carboxylic acid tert-butyl ester, 3-piperidine, 3-piperazine-1-carboxylic acid methyl ester, 3-piperidine-1-carboxylic acid tert-butylester, 1-piperidine-3-carboxylic acid methyl ester, or 1-piperidine-3-carboxylic acid tert-butyl ester; or a pharmaceutically acceptable salt thereof.

In an embodiment, the A_{2A} agonist is a compound of formula (Ia) wherein:

R¹ is each independently hydrogen or OH;

R² is hydrogen;

Z is selected from the group consisting of:

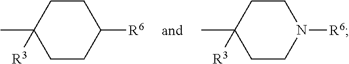

R³ is hydrogen or OH;

R⁶ is —CO₂R^{a};

R^{a} is (C₁-C₈)alkoxy, (C₃-C₆)cycloalkyl, (C₃-C₆)cycloalkyl-(C₁-C₃)alkylene, heterocycle, or heterocycle-(C₁-C₃)alkylene;

wherein any of the alkyl, cycloalkyl, heterocycle, aryl, or heteroaryl groups of R^{a} is unsubstituted or substituted on carbon with 1 or 2 substituents selected from the group consisting of halo, OR^{e}, (C₁-C₄)alkyl, —CN, NO₂, trifluoromethyl, trifluoromethoxy, CO₂R³, NR^{e}R^{e}, and C(O)NR^{e}R^{e}; and,

R^{e} is each independently H or (C₁-C₄)alkyl.

In an embodiment, the A_{2A} agonist is a compound of formula (Ia) wherein

Z is CR³R⁴R⁵; each R¹, R² and R³ is hydrogen; R⁴ and R⁵ together with the carbon atom to which they are attached form a cycloalkyl ring having 3, 4, 5, 6, 7, 8, 9 or 10 ring atoms; and

wherein the ring including R⁴ and R⁵ is substituted with —(CH₂)₀₋₆—Y; where Y is —CH₂OR^{a}, —CO₂R^{a}, —OC(O)R^{a}, —CH₂OC(O)R^{a}, —C(O)NR^{b}R^{c}, —CH₂SR^{a}, —C(S)OR^{a}, —OC(S)R^{a}, —CH₂OC(S)R^{a} or C(S)NR^{b}R^{c} or CH₂N(R^{b})(R^{a});

each R⁷ is independently hydrogen, (C₁-C₈)alkyl, (C₃-C₈)cycloalkyl, aryl or aryl(C₁-C₈)alkylene;

X is —CH₂OR^{a}, —CO₂R^{a}, —CH₂OC(O)R^{a}, —C(O)NR^{b}R^{c}, —CH₂SR^{a}, —C(S)OR^{a}, —CH₂OC(S)R^{a}, C(S)NR^{b}R^{c} or —CH₂N(R^{b})(R^{c});

each R^{a}, R^{b} and R^{c} is independently hydrogen, (C₁-C₈)alkyl, or (C₁-C₈)alkyl substituted with 1-3 (C₁-C₈)alkoxy, (C₃-C₈)cycloalkyl, (C₁-C₈)alkylthio, amino acid, aryl, aryl(C₁-C₈)alkylene, heteroaryl, or heteroaryl(C₁-C₈)alkylene; or R^{b} and R^{c}, together with the nitrogen to which they are attached, form a pyrrolidino, piperidino, morpholino, or thiomorpholino ring; and m is 0 to about 6; or a pharmaceutically acceptable salt thereof.

In an embodiment, —N(R⁷)₂ is amino, monomethylamino or cyclopropylamino.

In an embodiment, Z is carboxy- or —(C₁-C₄)alkoxycarbonyl-cyclohexyl(C₁-C₄)alkyl.

In an embodiment, R^{a} is H or (C₁-C₄)alkyl, i.e., methyl or ethyl.

In an embodiment, R^{b} is H, methyl or phenyl.

In an embodiment, R^{c} is H, methyl or phenyl.

In an embodiment, —(CR¹R²)ₘ— is —CH₂— or —CH₂—CH₂—.

In an embodiment, X is CO₂R^{a}, (C₂-C₅)alkanoylmethyl or amido.

In an embodiment, m is 1.

Specific compounds useful for practicing the methods herein are compounds JR3259, JR3269, JR4011, JR4009, JR-1085 and JR4007.

Specific A_{2A} adenosine receptor agonists useful in the methods herein having formula (Ia) include those described in U.S. Pat. No. 6,232,297.

Specific compounds of formula (Ia) are those wherein each R⁷ is H, X is ethylaminocarbonyl, R¹ is H, R² is H, m is 1, and either Z is 4-carboxycyclohexylmethyl (DWH-146a), Z is 4-methoxycarbonylcyclohexylmethyl (DWH-146e), Z is 4-isopropyl-carbonylcyclohexylmethyl (AB-1), Z is 4-acetoxymethyl-cyclohexylmethyl (JMR-193), Z is 4-pyrrolidine-1-carbonylcyclohexylmethyl (AB-3), or Z is 4-piperidine-1-carboxylic acid methyl ester (JR-1085), which is shown below.

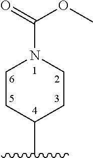 [SMILES: *C1CCN(C(=O)OC)CC1]

### Z=4-piperidine-1-carboxylic acid

Other embodiments are depicted below:

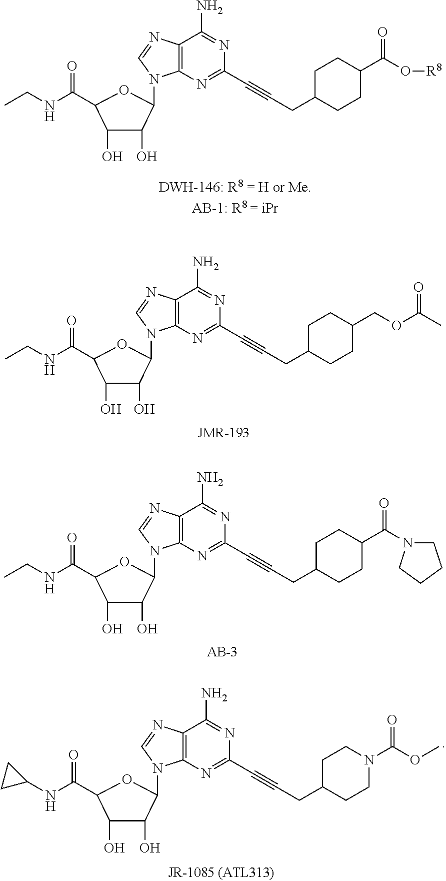

In an embodiment, ATL313 provides a better treatment for neuroinflammatory diseases (e.g. MS) compared to other A_{2A} agonists, such as CGS21680. In an embodiment, ATL313 is at least about 10%, 20%, 30%, 40%, 50%, 60%, 70%, 80%, 90%, 100%, or 150% better treatment of neuroinflammatory diseases (e.g., multiple sclerosis) compared to other A_{2A} agonists herein (e.g. CGS21680). In an embodiment, ATL313 is at least 2, 3, 4, 5, 10, or 20 times better for treatment of neuroinflammatory diseases (e.g., multiple sclerosis) compared to other A_{2A} agonists such as CGS21680. In an embodiment, ATL313 may provide a better treatment by having reduced side effects, better efficacy, increased safety, increased chemical stability, increased delay of symptom onset or worsening, increased survival rate, improved motor function, decreased pain, more selective, better binding properties (i.e., Ki), more bioavailability, increased duration of action, higher stability, and/or greater functional potency. In an embodiment, ATL313 may provide a better treatment by having treating or reducing the symptoms of multiple sclerosis, by treating MS pain, by improving motor function, by stabilizing motor function, by increasing or improving survival rate, by increasing remyelination, by decreasing or preventing demyelination, by sparing neuronal death, and/or by suppressing motor paralysis.

Exemplary compounds useful in the methods and compositions described herein are shown in Table 1 below.

TABLE 1

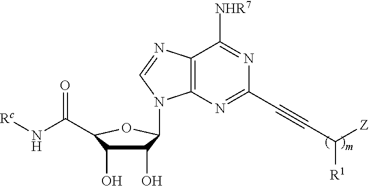

| Ex. | | | |
|---|---|---|---|
| # | R^{c} | R⁷ | —(CHR¹)ₘ—Z |
| 1. | Et | H | 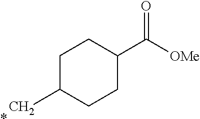 |
| 2. | Et | H | 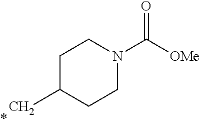 |
| 3. | cPr | H | 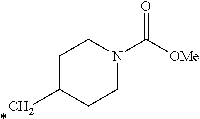 |
| 4. | Et | H | 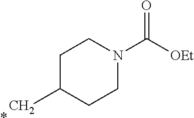 |
| 5. | cPr | H | 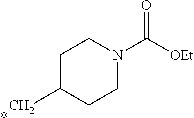 |
| 6. | Et | H | 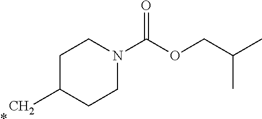 |
| 7. | cPr | H | 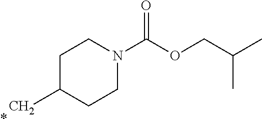 |
| 8. | Et | H | 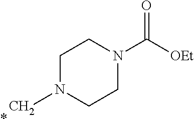 |
| 9. | Et | H | 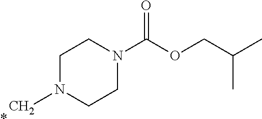 |
| 10. | Et | H | 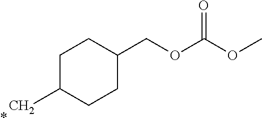 |
| 11. | Et | H | 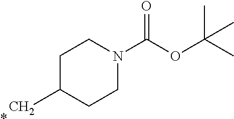 |
| 12. | cPr | H | 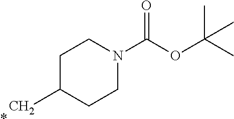 |
| 13. | Et | H | 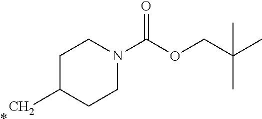 |
| 14. | cPr | H | 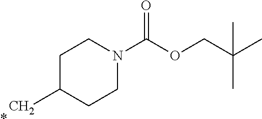 |
| 15. | Et | H | 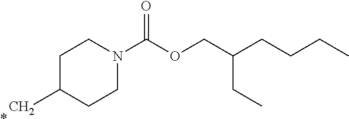 |
| 16. | cPr | H | 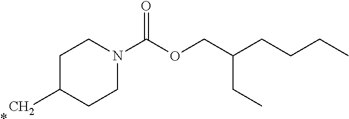 |
| 17. | cPr | H | 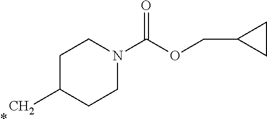 |
| 18. | Et | H | 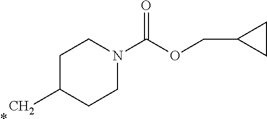 |
| 19. | cPr | H | 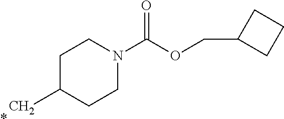 |
| 20. | Et | H | 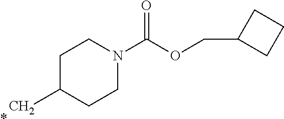 |
| 21. | cPr | H | 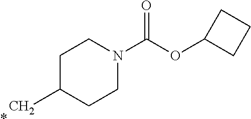 |
| 22. | Et | H | 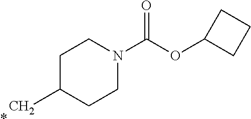 |
| 23. | Et | H | 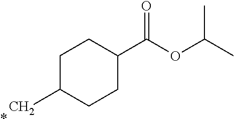 |
| 24. | cPr | H | 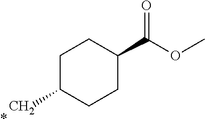 |
| 25. | Et | H | 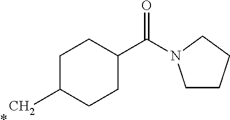 |
| 26. | Et | H | 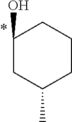 |
| 27. | Et | H | 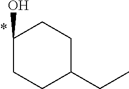 |
| 28. | Et | H | 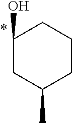 |
| 29. | Et | H | 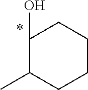 |
| 30. | Et | H | 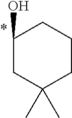 |
| 31. | cPr | H | 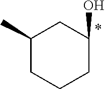 |
| 32. | Et | H | 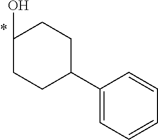 |
| 33. | Et | H | 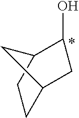 |
| 34. | cPr | H | 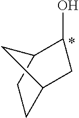 |
| 35. | cPr | H | 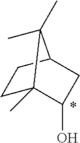 |
| 36. | Et | H | 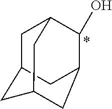 |
| 37. | cPr | H | 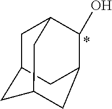 |
| 38. | Et | H | 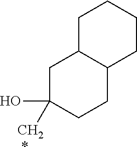 |
| 39. | cPr | H | 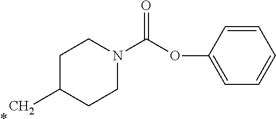 |
| 40. | Et | H | 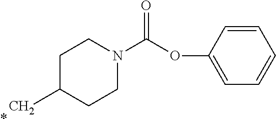 |
| 41. | cPr | H | 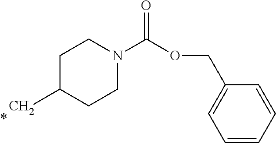 |
| 42. | Et | H | 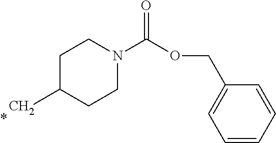 |

* signifies the point of attachment.

Additional embodiments of A_{2A} agonist are compounds of formula (Ib), (Ic), (Id) or a pharmaceutically acceptable salt thereof:

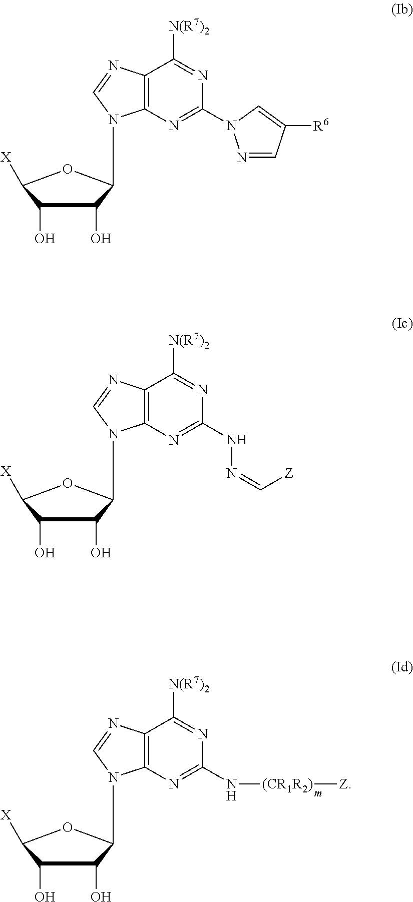

In an embodiment, the A_{2A} agonist is a compound of formula (Ie) wherein each of R⁷ is H, X is —C(O)NR^{b}R^{c} wherein R^{b} is hydrogen, and R^{c} is a C₂ alkyl (i.e. X is ethylaminocarbonyl), and R¹ and R² are each hydrogen, and Z is a 1-piperidyl-4-carboxylic acid or ester group, wherein R^{a} is hydrogen, methyl, ethyl, propyl, isopropyl, or t-butyl:

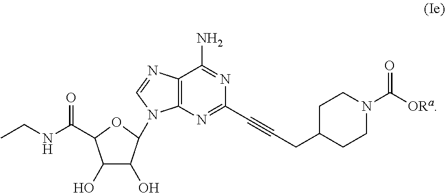

In an embodiment, the A_{2A} agonist is a compound of formula (If):

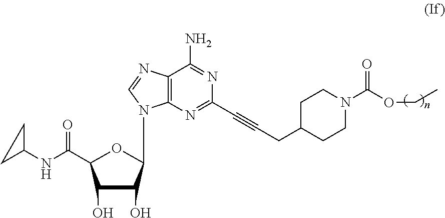

wherein n is 0, 1, 2, 3, 4, 5, 6, 7, 8, 9, 10, 11, 12, 13, 14, 15, 16, 17, or 18. In an embodiment, n is 5, 6, 7, 8, 9, 10, 11, 12, 13, 14, 15, 16, 17, or 18.

In an embodiment, the A_{2A} agonist is

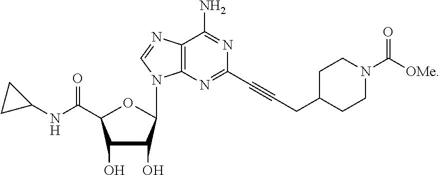

In an embodiment, the A_{2A} agonist is a compound of formula (Ig):

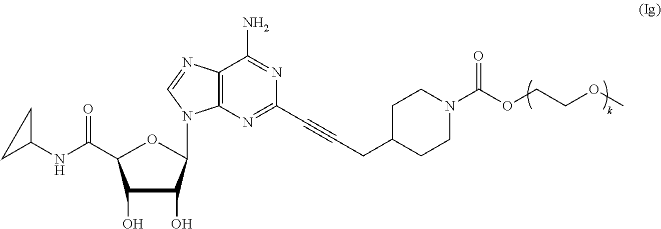

wherein k is 1, 2, 3, 4, 5, 6, 7, 8, 9, 10, 11, 12, 13, 14, 15, 16, 17, or 18.

In an embodiment, the A_{2A} agonist is a compound of formula (Ih):

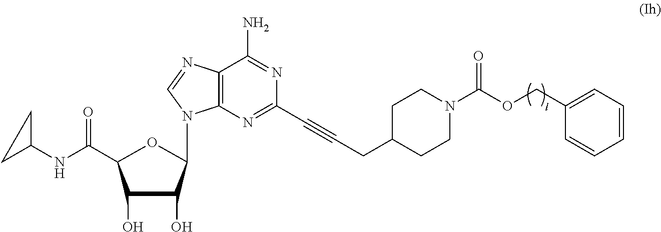

wherein l is 0, 1, 2, 3, or 4.

Additional examples of compounds include:

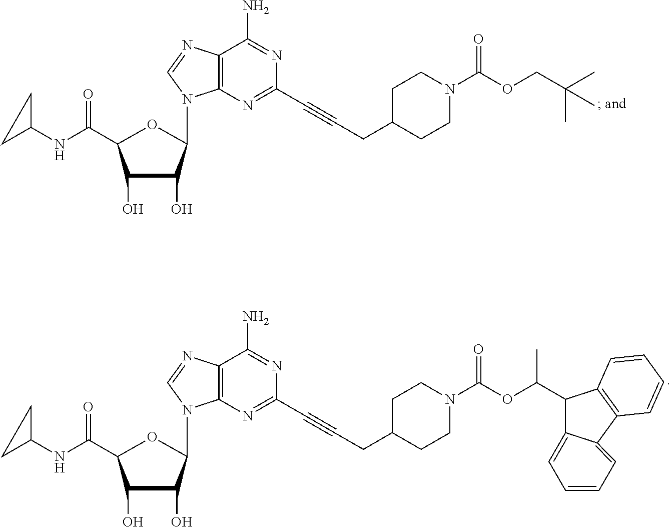

Additional examples of compounds useful in the methods and compositions described herein are illustrated in tables 3, 4, and 5 below:

TABLE 3

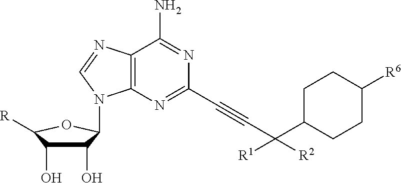

| Compound | R | R¹ | R² | R⁶ |
|---|---|---|---|---|
| ATL2037 | NECA | H | H | CH₂OH |
| MP9056 | NECA | OH | H | CH₂OH |
| ATL146a | NECA | H | H | CO₂H |
| MP9057 | NECA | OH | H | CO₂H |
| ATL146e | NECA | H | H | CO₂Me |
| MP9058 | NECA | OH | H | CO₂Me |
| JR2145 | CH₂OH | H | H | CO₂Me |
| MP9059 | CH₂OH | OH | H | CO₂Me |
| ATL193 | NECA | H | H | CH₂OAc |
| MP9060 | NECA | OH | H | CH₂OAc |
| JR2147 | CH₂OH | H | H | CH₂OAc |
| MP9061 | CH₂OH | OH | H | CH₂OAc |
| JR3023 | NECA | H | H | CH₂N(CH₃)₂ |
| MP9062 | NECA | OH | H | CH₂N(CH₃)₂ |
| JR3021 | NECA | H | H | COOCH₂CH₂NHBoc |
| MP9063 | NECA | OH | H | COOCH₂CH₂NHBoc |
| JR3033 | NECA | H | H | COOCH₂CH₂NH₂ |
| MP9064 | NECA | OH | H | COOCH₂CH₂NH₂ |
| JR3037 | NECA | H | H | CONHCH₂CH₃ |
| MP9065 | NECA | OH | H | CONHCH₂CH₃ |
| JR3055 | NECA | H | H | CONH₂ |
| MP9072 | NECA | OH | H | CONH₂ |
| JR3065 | NECA | H | H | CONHMe |
| MP9066 | NECA | OH | H | CONHMe |
| JR3067B | NECA | H | H | Me, cis CO₂Me |
| MP9067 | NECA | OH | H | Me, cis CO₂Me |
| JR3067A | NECA | H | H | Me, trans CO₂Me |
| MP9068 | NECA | OH | H | Me, trans CO₂Me |
| JR3087 | NECA | H | H | CH₂CH₃ |
| MP9069 | NECA | OH | H | CH₂CH₃ |
| JR3159A | NECA | OH | H | H |
| JR3159B | NECA | OH | H | H |
| JR3119 | NECA | H | H | COCH₃ |
| MP9070 | NECA | OH | H | COCH₃ |
| JR3121 | NECA | H | H | CHCH₃(OH) |
| MP9071 | NECA | OH | H | CHCH₃(OH) |
| JR3139 | NECA | OH | C₆H₁₁ | H |

NECA = CH₃CH₂N(H)C(O)—

TABLE 4

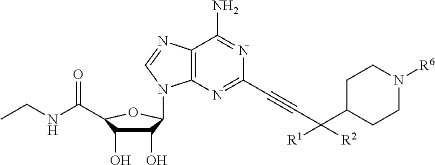

| Compound | R¹ | R² | R⁶ |
|---|---|---|---|
| JR3261 | H | H | H |
| JR3259 | H | H | CO₂tBu |
| JR3269 | H | H | CO₂Et |
| JR4011 | H | H | CO₂iBu |
| JR4009 | H | H | CO₂iPr |
| JR4007 | H | H | COMe |
| JR4051 | H | H | COC(CH₃)₃ |
| JR4047 | H | H | COCH₂(CH₃)₃ |
| MP9047 | H | H | COCH₃ |
| MP9048 | H | H | C(O)N(CH₃)₂ |
| MP9049 | H | H | C(O)N(CH₃)Et |
| MP9050 | H | H | C(O)N(CH₃)iPr |
| MP9051 | H | H | C(O)N(CH₃)iBu |
| MP9052 | H | H | C(O)NH(CH₃) |
| MP9053 | H | H | C(O)NH(Et) |
| MP9054 | H | H | C(O)NH(iPr) |
| MP9055 | H | H | C(O)NH(iBu) |
| TX3261 | OH | H | H |
| TX3259 | OH | H | CO₂tBu |
| TX3269 | OH | H | CO₂Et |
| TX4011 | OH | H | CO₂iBu |
| TX4009 | OH | H | CO₂iPr |
| TX4007 | OH | H | COMe |
| TX4051 | OH | H | COC(CH₃)₃ |
| TX4047 | OH | H | COCH₂(CH₃)₃ |
| TX9047 | OH | H | COCH₃ |
| TX9048 | OH | H | C(O)N(CH₃)₂ |
| TX9049 | OH | H | C(O)N(CH₃)Et |
| TX9050 | OH | H | C(O)N(CH₃)iPr |
| TX9051 | OH | H | C(O)N(CH₃)iBu |
| TX9052 | OH | H | C(O)NH(CH₃) |
| TX9053 | OH | H | C(O)NH(Et) |
| TX9054 | OH | H | C(O)NH(iPr) |
| TX9055 | OH | H | C(O)NH(iBu) |

TABLE 5

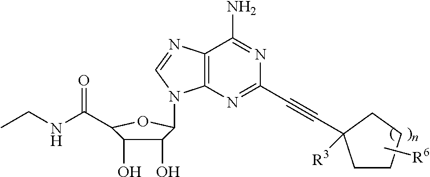

| Compound | n | R³ | R⁶ |
|---|---|---|---|
| JR3135 | 1 | OH | H |
| JR3089 | 2 | OH | H |
| JR3205 | 2 | NH₂ | H |
| JR3177A | 2 | OH | 2-CH₃ |
| JR3177B | 2 | OH | 2-CH₃ |
| JR3181A | 2 | OH | 2-CH₃ |
| JR3181B | 2 | OH | 2-CH₃ |
| JR3227 | 2 | OH | 2-C(CH₃)₃ |
| JR9876 | 2 | OH | 2-C₆H₅ |
| JR3179 | 2 | OH | 3-CH₃ |
| JR3221 | 2 | OH (R) | 3-CH₃ (R) |
| ATL 203 | 2 | OH (S) | 3-CH₃ (R) |
| MP9041 | 2 | OH (R) | 3-CH₃ (S) |
| MP9042 | 2 | OH (S) | 3-CH₃ (S) |
| JR3201B | 2 | OH | 3-(CH₃)₂ |
| MP9043 | 2 | OH (R) | 3-CH₂CH₃ (R) |
| MP9044 | 2 | OH (S) | 3-CH₂CH₃ (R) |
| MP9045 | 2 | OH (R) | 3-CH₂CH₃ (S) |
| MP9046 | 2 | OH (S) | 3-CH₂CH₃ (S) |
| JR3163 | 2 | OH | 3-(CH₃)₂, 5-(CH₃)₂ |
| JR9875 | 2 | OH | 4-CH₃ |
| JR3149 | 2 | OH | 4-C₂H₅ |
| JR3203 | 2 | OH | 4-C(CH₃)₃ |
| JR3161 | 2 | OH | 4-C₆H₅ |

Additional examples of A_{2A} adenosine receptor agonists of formula (II) that are expected to be useful in the present invention include those described in U.S. Pat. No. 6,232,297. These compounds, having formula (II), can be prepared according to the methods described therein.

Further examples of A_{2A} agonists that are useful in the methods and compositions provided herein include compounds of formula II or a stereoisomer or pharmaceutically acceptable salt thereof:

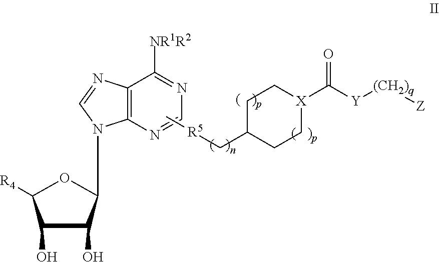

wherein:

R¹ and R² independently are selected from the group consisting of H, (C₁-C₈)alkyl, (C₃-C₈)cycloalkyl, (C₃-C₈)cycloalkyl(C₁-C₈)alkylene, aryl, aryl(C₁-C₈)alkylene, heteroaryl, heteroaryl(C₁-C₈)alkylene-, diaryl(C₁-C₈)alkylene, and diheteroaryl(C₁-C₈)alkylene, wherein the aryl and heteroaryl rings are optionally substituted with 1-4 groups independently selected from fluoro, chloro, iodo, bromo, methyl, trifluoromethyl, and methoxy;

each R independently is selected from the group consisting of H, C₁-C₄ alkyl, cyclopropyl, cyclobutyl, and (CH₂)ₐcyclopropyl;

X is CH or N, provided that when X is CH then Z cannot be substituted with halogen, C₁-C₆ alkyl, hydroxyl, amino, or mono- or di-(C₁-C₆-alkyl)amino;

Y is selected from the group consisting of O, NR¹, —(OCH₂CH₂O)ₘCH₂—, and —(NR¹CH₂CH₂O)ₘCH₂—, provided that when Y is O or NR¹, then at least one substituent is present on Z;

Z is selected from the group consisting of 5-membered heteroaryl, 6-membered aryl, 6-membered heteroaryl, carbocyclic biaryl, and heterocyclic biaryl, wherein the point of attachment of Y to Z is a carbon atom on Z, wherein Z is substituted with 0-4 groups independently selected from the group consisting of F, Cl, Br, I, (C₁-C₄)alkyl, —(CH₂)ₐOR³, —(CH₂)ₐNR³R³, —NHOH, —NR³NR³R³, nitro, —(CH₂)ₐCN, —(CH₂)ₐCO₂R³, —(CH₂)ₐCONR³R³, trifluoromethyl, and trifluoromethoxy;

alternatively, Y and Z together form an indolyl, indolinyl, isoindolinyl, tetrahydroisoquinolinyl, or tetrahydroquinolinyl moiety wherein the point of attachment is via the ring nitrogen and wherein said indolyl, indolinyl, isoindolinyl, tetrahydroisoquinolinyl, or tetrahydroquinolinyl moiety, which is substituted with 0-4 groups independently selected from the group consisting of F, Cl, Br, I, C₁-C₄ alkyl, —(CH₂)ₐOR³, —(CH₂)ₐNR³R³, —NHOH, —NR³NR³R³, NO₂, —(CH₂)ₐCN, —(CH₂)ₐCO₂R³, —(CH₂)ₐCONR³R³, CF₃, and OCF₃;

R³ is independently selected from the group consisting of H, (C₁-C₆)alkyl, cycloalkyl, aryl, and heteroaryl;

R⁴ is selected from the group consisting of CH₂OR, C(O)NRR, and CO₂R;

R⁵ is selected from the group consisting of CH₂CH₂, CH═CH, and CC;

a is selected from 0, 1, and 2;

m is selected from 1, 2, and 3;

n is selected from 0, 1, and 2;

each p independently is selected from 0, 1, and 2; and,

q is selected from 0, 1, and 2.

In an embodiment, the A_{2A} agonist is a compound of formula II wherein R⁵ is C≡C and has a structure of formula IIa or a pharmaceutically acceptable salt thereof:

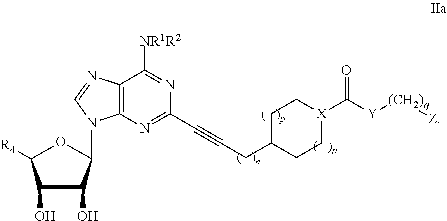

In an embodiment, the A_{2A} agonist is a compound of formula II wherein R⁵ is C≡C, n is 1, p is 1, X is N, Y is O, and has a structure of formula IIb or a pharmaceutically acceptable salt thereof:

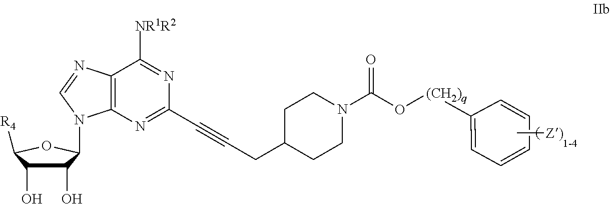

wherein:

each Z′ is independently selected from the group consisting F, Cl, Br, I, C₁-C₄ alkyl, —(CH₂)ₐOR³, —(CH₂)ₐNR³R³, —NHOH, —NR³NR³R³, NO₂, —(CH₂)ₐCN, —(CH₂)ₐCO₂R³, —(CH₂)ₐCONR³R³, CF₃, and OCF₃.

In an embodiment, R is selected from H, methyl, ethyl or cyclopropyl.

In an embodiment, the A_{2A} agonist is a compound of formula II wherein R⁵ is C≡C, n is 1, p is 1, X is N, Y is O and has a structure of formula IIc or a pharmaceutically acceptable salt thereof:

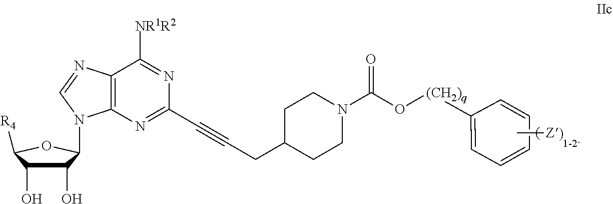

In an embodiment, Z′ is selected from the group consisting of F, Cl, methyl, OR³, NO₂, CN, NR³R³ and CO₂R³.

In an embodiment, R³ is methyl or hydrogen.

Additional exemplary compounds that are useful in the provided methods and compositions herein are shown in Table 2.

| | |
|---|---|
| | i |
| 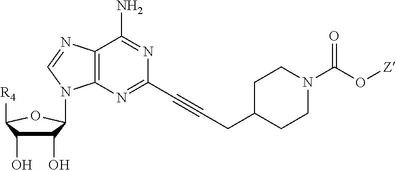 | |
| | ii |
| 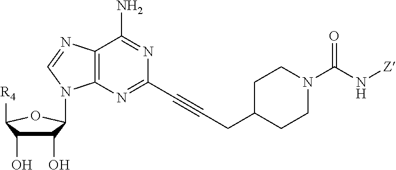 | |
| | iii |
| 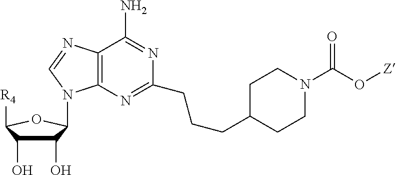 | |

| Ex. # | R⁴ | Z′ |
|---|---|---|
| 1 | C | 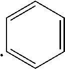 |
| 2 | C | 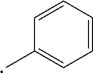 |
| 3 | C | 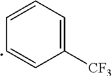 |
| 4 | A | 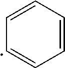 |
| 5 | C | 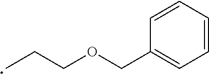 |
| 6 | A | 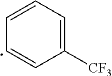 |
| 7 | A | 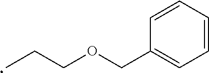 |
| 8 | C | 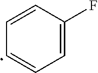 |
| 9 | C | 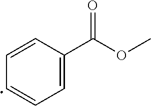 |
| 10 | C | 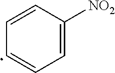 |
| 11 | A | 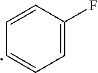 |
| 12 | A | 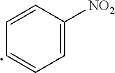 |
| 13 | A | 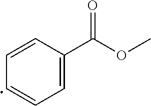 |
| 14 | C | 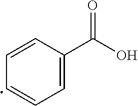 |
| 15 | B | 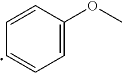 |
| 16 | B | 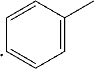 |
| 17 | C | 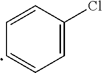 |
| 18 | C | 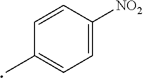 |
| 19 | B | 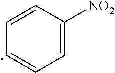 |
| 20 | C | 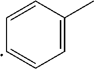 |
| 21 | C | 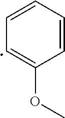 |
| 22 | C | 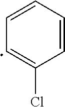 |
| 23 | C | 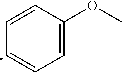 |
| 24 | B | 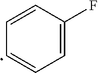 |
| 25 | B | 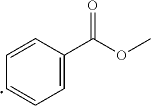 |
| 26 | B | 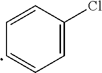 |
| 27 | A | 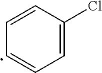 |
| 28 | A | 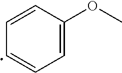 |
| 29 | A | 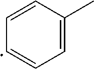 |
| 30 | A | 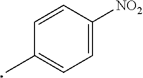 |
| 31 | B | 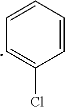 |
| 32 | B | 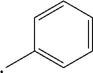 |
| 33 | B | 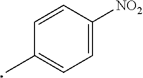 |
| 34 | B | 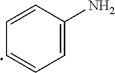 |
| 35 | A | 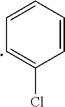 |
| 36 | A | 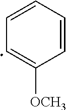 |
| 37 (iii) | B | 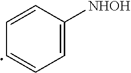 |
| 38 (iii) | C | 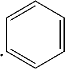 |
| 39 (iii) | C | 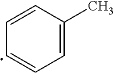 |
| 40 (iii) | C | 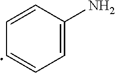 |
| 41 (iii) | C | 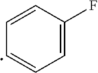 |
| 42 | C | 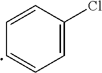 |
| 43 (ii) | C | 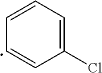 |
| 44 (ii) | A | 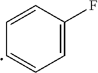 |
| 45 (ii) | A | 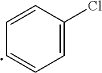 |
| 46 (ii) | A | 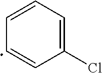 |
| 47 (ii) | C | 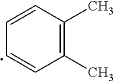 |
| 48 (ii) | C | 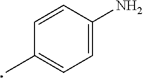 |
| 49 | B | 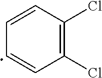 |
| 50 | B | 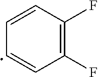 |
| 51 | C | 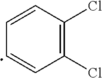 |
| 52 | C | 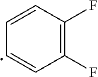 |
| 53 | A | 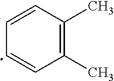 |
| 54 | A | 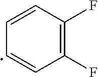 |
| 55 | A | 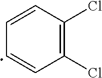 |
| 56 | C | 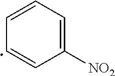 |
| 57 | C | 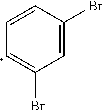 |

R⁴ = A: CH₂OH; B: C(O)NHEthyl; C: C(O)NHCyclopropyl.
Compounds are of formula (i), unless indicated.

Additional examples of A_{2A} adenosine receptor agonists useful in the methods herein include those described in U.S. Pat. No. 6,232,297 and in U.S. Patent Application No. 2003/0186926 A 1.

Another specific group of agonists of A_{2A} adenosine receptors that are useful for the methods and compositions provided herein include compounds of formula (III):

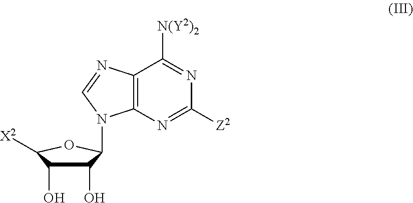

wherein Z² is a group selected from the group consisting of −OR¹², —NR¹³R¹⁴, a —C≡C—Z³, and —NH—N═R¹⁷;

each Y² is individually H, C₁-C₆ alkyl, C₃-C₇ cycloalkyl, phenyl or phenyl C₁-C₃ alkyl;

R¹² is C₁₋₄-alkyl; C₁₋₄alkyl substituted with one or more C₁₋₄-alkoxy groups, halogens (fluorine, chlorine or bromine), hydroxy groups, amino groups, mono(C₁₋₄-alkyl)amino groups, di(C₁₋₄-alkyl)amino groups or C₆₋₁₀-aryl groups wherein the aryl groups may be substituted with one or more halogens (fluorine, chlorine or bromine), C₁₋₄-alkyl groups, hydroxy groups, amino groups, mono(C₁₋₄-alkyl)amino groups or di(C₁₋₄-alkyl)amino groups); or C₆₋₁₀-aryl; or C₆₋₁₀-aryl substituted with one or more halogens (fluorine, chlorine or bromine), hydroxy groups, amino groups, mono(C₁₋₄-alkyl)amino groups, di(C₁₋₄-alkyl)amino groups or C₁₋₄-alkyl groups;

one of R¹³ and R¹⁴ has the same meaning as R¹² and the other is hydrogen; and

R¹⁷ is a group having the formula (A)

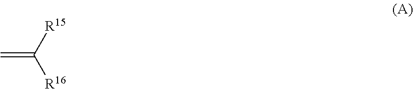

wherein each of R¹⁵ and R¹⁶ independently may be hydrogen, (C₃-C₇)cycloalkyl or any of the meanings of R¹², provided that R¹⁵ and R¹⁶ are not both hydrogen;

X² is CH₂OH, CH₃, CO₂R²⁰ or C(═O)NR²¹ wherein R²⁰ has the same meaning as R¹³ and wherein R²¹ and R²² have the same meanings as R¹⁵ and R¹⁶ or R²¹ and R²² are both H;

Z³ has one of the following meanings:

C₆-C₁₀ aryl, optionally substituted with one to three halogen atoms, C₁-C₆ alkyl, C₁-C₆ haloalkyl, C₁-C₆ alkoxy, C₁-C₆ haloalkoxy, C₂-C₆ alkoxycarbonyl, C₂-C₆ alkoxyalkyl, C₁-C₆ alkylthio, thio, CHO, cyanomethyl, nitro, cyano, hydroxy, carboxy, C₂-C₆ acyl, amino C₁-C₃ monoalkylamino, C₂-C₆ dialkylamino, methylenedioxy or aminocarbonyl;

a group of formula —(CH₂)_{q}-Het wherein q is 0 or an integer from 1 to 3 and Het is 5 or 6 membered heterocyclic aromatic or non-aromatic ring, optionally benzocondensed, containing 1 to 3 heteroatoms selected from non-peroxide oxygen, nitrogen or sulphur, linked through a carbon atom or through a nitrogen atom;

C₃-C₇ cycloalkyl optionally containing unsaturation or C₂-C₄ alkenyl;

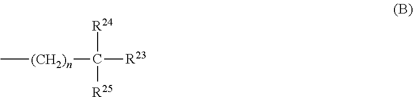

wherein

R²³ is hydrogen, methyl or phenyl;

R²⁴ is hydrogen, C₁-C₆ linear or branched alkyl, C₅-C₆ cycloalkyl or C₃-C₇ cycloalkenyl, phenyl-C₁-C₂-alkyl or R²³ and R²⁴, taken together, form a 5 or 6-membered carbocyclic ring or R²⁵ is hydrogen and R²³ and R²⁴, taken together, form an oxo group or a corresponding acetalic derivative;

R²⁵ is OH, NH₂ dialkylamino, halogen, cyano; and n is 0 or 1 to 4;

or C₁-C₁₆ alkyl, optionally including 1-2 double bonds, O, S or NY²;

or a pharmaceutically acceptable salt thereof.

Specific C₆₋₁₀-aryl groups include phenyl and naphthyl.

Additional embodiments include compounds of formula (III), wherein Z² is a group of the formula (C)

—O—(CH₂)ₙ—Ar (C)

wherein n is an integer from 1-4, e.g., 2, and Ar is a phenyl group, tolyl group, naphthyl group, xylyl group or mesityl group. In an embodiment, Ar is a para-tolyl group and n is 2.

Additional embodiments include compounds of formula (III), wherein Z² is a group of formula (D)

NH—N═CHCy (D)

wherein Cy is a C₃₋₇-cycloalkyl group, such as cyclohexyl or a C₁₋₄ alkyl group, such as isopropyl.

Additional embodiments include compounds of formula (III), Z² is a group of the formula (E)

C≡C—Z³ (E)

wherein Z³ is C₃-C₁₆ alkyl, hydroxy C₂-C₆ alkyl or (phenyl) (hydroxymethyl).

Additional examples of compounds of formula (III) include those shown below:

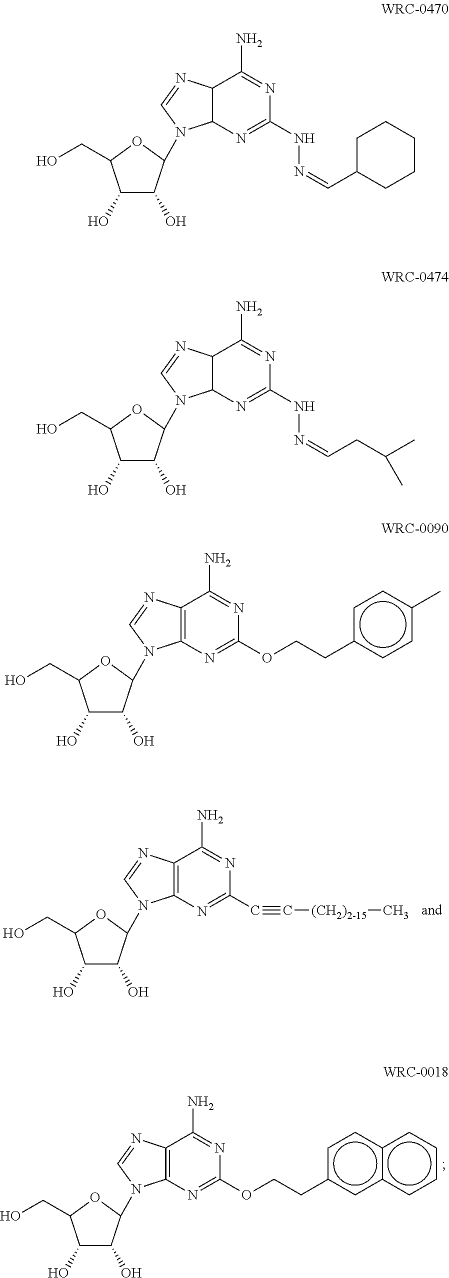

wherein the H on CH₂OH can optionally be replaced by ethylaminocarbonyl.

Such compounds may be synthesized as described in: Olsson et al. (U.S. Pat. Nos. 5,140,015 and 5,278,150); Cristalli (U.S. Pat. No. 5,593,975); Miyasaka et al. (U.S. Pat. No. 4,956,345); Hutchinson, A. J. et al., J. Pharmacol. Exp. Ther., 251, 47 (1989); Olsson, R. A. et al., J. Med. Chem., 29, 1683 (1986); Bridges, A. J. et al., J. Med. Chem., 31, 1282 (1988); Hutchinson, A. J. et al., J. Med. Chem., 33, 1919 (1990); Ukeeda, M. et al., J. Med. Chem., 34, 1334 (1991); Francis, J. E. et al., J. Med. Chem., 34, 2570 (1991); Yoneyama, F. et al., Eur. J. Pharmacol., 213, 199-204 (1992); Peet, N. P. et al., J. Med. Chem., 35, 3263 (1992); and Cristalli, G. et al., J. Med. Chem., 35, 2363 (1992); all of which are incorporated herein by reference.

Another embodiment includes a compound of formula (III) wherein Z² is a group having formula (F):

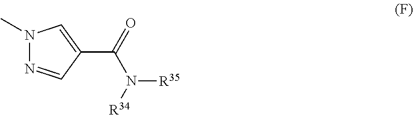

wherein R³⁴ and R³⁵ are independently H, C₁-C₆ alkyl, C₃-C₇ cycloalkyl, phenyl, phenyl C₁-C₃ alkyl or R³⁴ and R³⁵ taken together with the nitrogen atom are a 5- or 6-membered heterocyclic ring containing 1-2 heteroatoms selected from non-peroxide oxygen, N(R¹³), or sulphur atoms. In an embodiment, one of R³⁴ and R³⁵ is hydrogen and the other is ethyl, methyl or propyl. In an embodiment, one of R³⁴ and R³⁵ is hydrogen and the other is ethyl or methyl.

A pyrazole derivative useful for the methods and compositions provided herein is a compound having the formula:

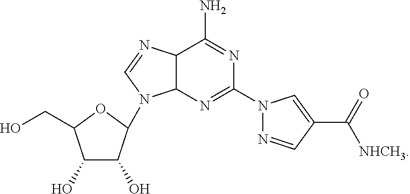 [SMILES: CNC(=O)c1cnn(C2=NC3C(N=CN3C3OC(CO)[C@@H](O)[C@H]3O)C(N)=N2)c1]

Another specific group of agonists of A_{2A} adenosine receptors that are useful in the methods provided herein include compounds having the formula (IV):

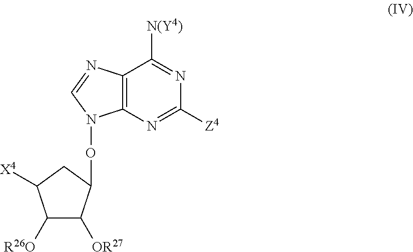

wherein Z⁴ is —NR²⁸R²⁹;

R²⁸ is hydrogen or (C₁-C₄) alkyl; and R²⁹ is
a) (C₁-C₄) alkyl;
b) (C₁-C₄) alkyl substituted with one or more (C₁-C₄) alkoxy, halogen, hydroxy, amino, mono((C₁-C₄) alkyl)amino, di((C₁-C₄) alkyl)amino or (C₆-C₁₀) aryl wherein aryl is optionally substituted with one or more halogen, hydroxy, amino, (C₁-C₄)alkyl, R³⁰OOC—((C₁-C₄)alkyl)-, R³¹R³²NC(═O)—((C₁-C₄)alkyl)-, mono((C₁-C₄)alkyl)amino or di((C₁-C₄)alkyl)amino;
c) (C₆-C₁₀)aryl; or
d) (C₆-C₁₀)aryl substituted with one or more halogen, hydroxy, amino, mono((C₁-C₄)alkyl)amino, di((C₁-C₄)alkyl)amino or (C₁-C₄)alkyl;

wherein each Y⁴ is individually H, (C₁-C₆)alkyl, (C₃-C₇)cycloalkyl, phenyl or phenyl(C₁-C₃)alkyl; and X⁴ is —C(═O)NR³¹R³², —COOR³⁰, or —CH₂OR³⁰;

wherein each of R³¹ and R³² are independently; hydrogen; C₃₋₄-cycloalkyl; (C₁-C₄)alkyl; (C₁-C₄)alkyl substituted with one or more (C₁-C₄)alkoxy, halogen, hydroxy, —COOR³³, amino, mono((C₁-C₄)alkyl)amino, di((C₁-C₄)alkyl)amino or (C₆-C₁₀)aryl wherein aryl is optionally substituted with one or more halogen, (C₁-C₄)alkyl, hydroxy, amino, mono((C₁-C₄) alkyl)amino or di((C₁-C₄) alkyl)amino; (C₆-C₁₀)aryl; or (C₆-C₁₀)aryl substituted with one or more halogen, hydroxy, amino, mono((C₁-C₄)alkyl)amino, di((C₁-C₄)alkyl)amino or (C₁-C₄)alkyl;

R²⁶ and R²⁷ independently represent hydrogen, lower alkanoyl, lower alkoxy-lower alkanoyl, aroyl, carbamoyl or mono- or di-lower alkylcarbamoyl; and R³⁰ and R³³ are independently hydrogen, (C₁-C₄)alkyl, (C₆-C₁₀)aryl or (C₆-C₁₀)aryl((C₁-C₄)alkyl); or a pharmaceutically acceptable salt thereof.

Additional embodiments include compounds wherein at least one of R²⁸ and R²⁹ is (C₁-C₄)alkyl substituted with one or more (C₁-C₄)alkoxy, halogen, hydroxy, amino, mono((C₁-C₄)alkyl)amino, di((C₁-C₄)alkyl)amino or (C₆-C₁₀)aryl wherein aryl is optionally substituted with one or more halogen, hydroxy, amino, (C₁-C₄)alkyl, R³⁰OOC—(C₁-C₄)alkyl, mono((C₁-C₄)alkyl)amino or di((C₁-C₄)alkyl)amino.

Another embodiment includes compounds wherein at least one of R³¹ and R³² is C₁₋₄-alkyl substituted with one or more (C₁-C₄)alkoxy, halogen, hydroxy, amino, mono((C₁-C₄)alkyl)amino, di((C₁-C₄)alkyl)amino or C₆₋₁₀-aryl wherein aryl is optionally substituted with one or more halogen, hydroxy, amino, (C₁-C₄)alkyl, R³⁰OOC—(C₁-C₄)alkylene-, mono((C₁-C₄)alkyl)amino or di((C₁-C₄)alkyl)amino.

Another embodiment includes compounds wherein at least one of R²⁸ and R²⁹ is C₆₋₁₀-aryl substituted with one or more halogen, hydroxy, amino, mono((C₁-C₄)alkyl)amino, di((C₁-C₄)alkyl)amino or (C₁-C₄)alkyl.

Another embodiment includes compounds wherein at least one of R³¹ and R³² is C₆₋₁₀-aryl substituted with one or more halogen, hydroxy, amino, mono((C₁-C₄)alkyl)-amino, di((C₁-C₄)alkyl)amino or (C₁-C₄)alkyl.

Another embodiment includes compounds wherein R³¹ is hydrogen and R³² is (C₁-C₄)alkyl, cyclopropyl or hydroxy-(C₂-C₄)alkyl. A specific R²⁸ group is (C₁-C₄)alkyl substituted with (C₆-C₁₀)aryl, that is in turn substituted with R³⁰O(O)C—(C₁-C₄)alkylene-.

In an embodiment, a compound of formula (IV) is:

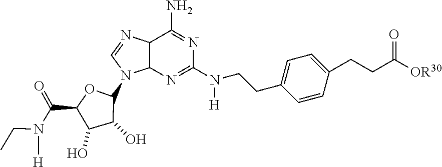

wherein R³⁰ is hydrogen, methyl, ethyl, n-propyl or isopropyl. An embodiment provides a compound wherein the R³⁰ group is methyl or ethyl. In an embodiment, the R³⁰ group is methyl.

Two other compounds that can be used in embodiments for the compositions or for practicing the methods herein have the formula:

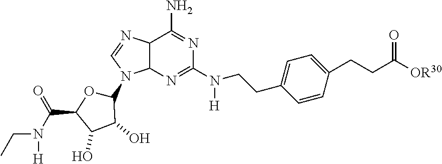

wherein R³⁰ is hydrogen (acid, CGS21680) and wherein R³⁰ is methyl (ester, JR2171).

The compounds of the embodiments described herein having formula (IV) may be synthesized as described in: U.S. Pat. No. 4,968,697 or J. Med. Chem., 33, 1919-1924, (1990).

In an embodiment, the A_{2A} agonist useful here is IB-MECA:

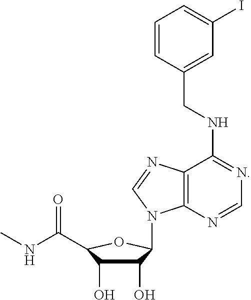

The compounds of formulas described herein, e.g., (I), (II), (III), and (IV), may have more than one chiral center and may be isolated in optically active and racemic forms. In an embodiment, the riboside moiety of the compounds is derived from D-ribose, i.e., the 3N,4N-hydroxyl groups are alpha to the sugar ring and the 2N and 5N groups is beta (3R,4S,2R,5S). When the two groups on the cyclohexyl group are in the 1- and 4-position, they may be trans. Some compounds may exhibit polymorphism. It is to be understood that the methods and compositions described herein encompass any racemic, optically-active, polymorphic, or stereoisomeric form, or mixtures thereof, of a compound described herein, which possess the useful properties described herein. It is well known in the art how to prepare optically active forms (for example, by resolution of the racemic form by recrystallization techniques, or enzymatic techniques, by synthesis from optically-active starting materials, by chiral synthesis, or by chromatographic separation using a chiral stationary phase) and how to determine adenosine agonist activity using the tests described herein, or using other similar tests which are well known in the art.

In one embodiment the A_{2A} agonist is ATL313 which has the following structure:

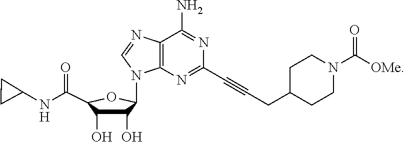

In one embodiment the A_{2A} agonist has the following structure:

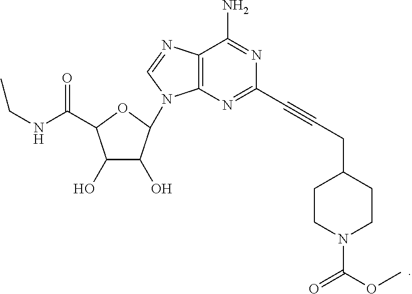 [SMILES: CCNC(=O)C1OC(n2cnc3c(N)nc(C#CCC4CCN(C(=O)OC)CC4)nc32)C(O)C1O]

In one embodiment the A_{2A} agonist has the following structure:

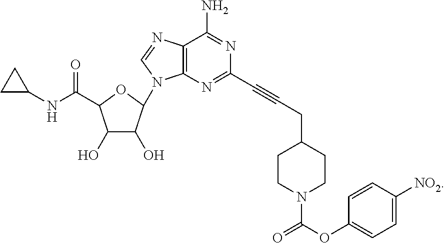 [SMILES: Nc1nc(C#CCC2CCN(C(=O)Oc3ccc([N+](=O)[O-])cc3)CC2)nc2c1ncn2C1OC(C(=O)NC2CC2)C(O)C1O]

In one embodiment the A_{2A} agonist has the following structure:

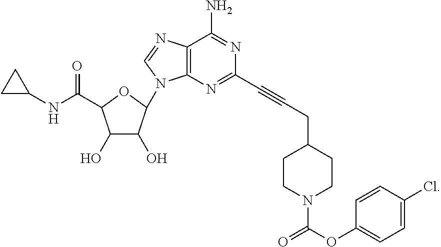 [SMILES: Nc1nc(C#CCC2CCN(C(=O)Oc3ccc(Cl)cc3)CC2)nc2c1ncn2C1OC(C(=O)NC2CC2)C(O)C1O]

In one embodiment the A_{2A} agonist has the following structure:

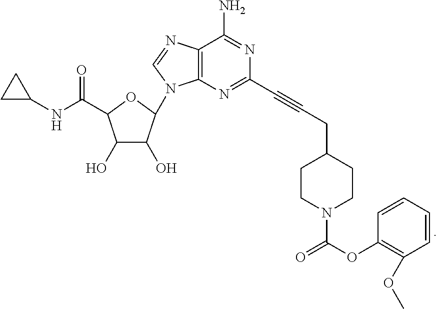 [SMILES: COc1ccccc1OC(=O)N1CCC(CC#Cc2nc(N)c3ncn(C4OC(C(=O)NC5CC5)C(O)C4O)c3n2)CC1]

In one embodiment the A_{2A} agonist has the following structure:

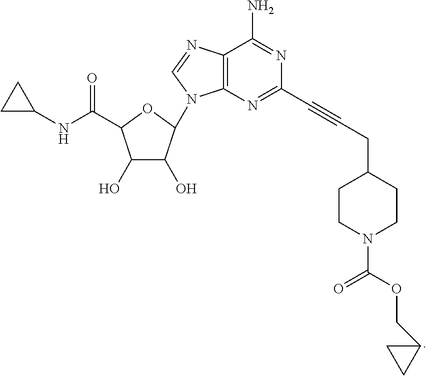 [SMILES: Nc1nc(C#CCC2CCN(C(=O)OCC3CC3)CC2)nc2c1ncn2C1OC(C(=O)NC2CC2)C(O)C1O]

In one embodiment the A_{2A} agonist has the following structure:

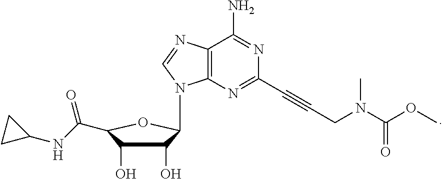

In one embodiment the A_{2A} agonist has the following structure:

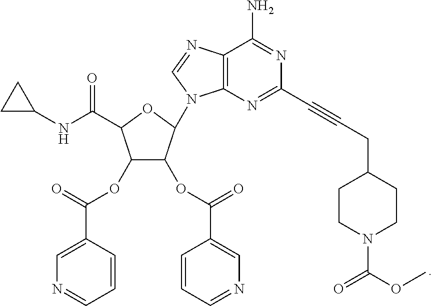 [SMILES: COC(=O)N1CCC(CC#Cc2nc(N)c3ncn(C4OC(C(=O)NC5CC5)C(OC(=O)c5cccnc5)C4OC(=O)c4cccnc4)c3n2)CC1]

In one embodiment the A_{2A} agonist has the following structure:

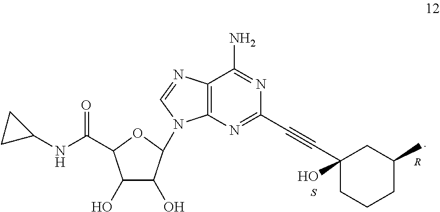

In one embodiment the A_{2A} agonist has the following structure:

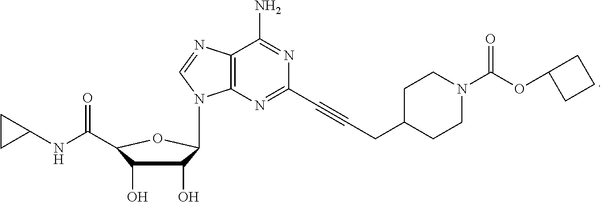

In one embodiment the A_{2A} agonist has the following structure:

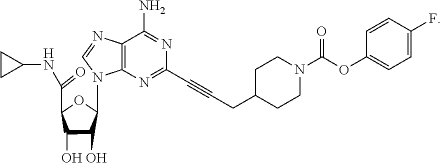

In one embodiment the A_{2A} agonist has the following structure:

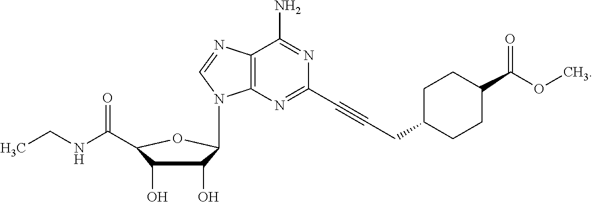

Unless defined otherwise, all technical and scientific terms used herein have the same meaning as is commonly understood by one of skill in the art.

Unless specified otherwise, all numbers contained herein are approximate values. For example, an embodiment described as having a survival rate of 100% may include survival rates of 100.02%, 100.5%, 99% and 103% and may not be limited to 100.00%.

As used herein, the singular form “a”, “an” and “the” includes plural references unless the context clearly dictates otherwise.

As used herein, “patient” includes one or more subjects, individuals, or mammals such as humans, non-human primates, murine, caprine, bovine, ovine, equine, porcine, canine, and feline.

As used herein, “intrathecal” refers to intrathecal drug administration by the injection of a therapeutic agent into the space or fluid surrounding the spinal cord, for example, into the subarachnoid space of the spinal cord. Catheters, syringe injections and devices, such as pumps, are non-limiting examples of intrathecal delivery devices.

As used herein, “A_{2A} agonist” refers to an agent that activates the Adenosine A_{2A} receptor (also known as A_{2A} adenosine receptor or A_{2A} receptor) with a Ki of <1 μM. An A_{2A} agonist may be selective for an A_{2A} receptor (e.g., at least a ratio of 10:1, 50:1, or 100:1) over another adenosine receptor subtype. An A_{2A} agonist may also be cross reactive with other adenosine receptor subtypes (e.g., A₁, A_{2B}, and A₃). The A_{2A} agonist may activate other receptors with a greater or lesser affinity than the A_{2A} receptor. An A_{2A} agonist is also known as an A_{2A} receptor agonist, A_{2A} adenosine receptor agonist or adenosine A_{2A} receptor agonist. Also contemplated for use herein are other agonists or agents, currently being developed or may be developed in the future, which have similar or equivalent properties, characteristics, structural features, or which are effectively or functionally equivalent. Other agents are useful if they perform the same function in the same way to yield substantially the same result.

As used herein, “halo” or alternatively, “halogen” is fluoro, chloro, bromo, or iodo. The terms “haloalkyl,” “haloalkenyl,” “haloalkynyl” and “haloalkoxy” include alkyl, alkenyl, alkynyl and alkoxy structures that are substituted with one or more halo groups or with combinations thereof. For example, the terms “fluoroalkyl” and “fluoroalkoxy” include haloalkyl and haloalkoxy groups, respectively, in which the halo is fluorine.

As used herein, “alkyl”, “alkoxy”, “aralkyl”, “alkylaryl”, etc. denote both straight and branched alkyl groups; but reference to an individual radical such as “propyl” embraces only the straight chain radical, a branched chain isomer such as “isopropyl” being specifically referred to.

As used herein, “aryl” denotes a phenyl radical or an ortho-fused bicyclic carbocyclic radical having about nine to ten ring atoms in which at least one ring is aromatic. Heteroaryl denotes a radical of a monocyclic aromatic ring containing five or six ring atoms consisting of carbon and one, two, three, or four heteroatoms each selected from the group consisting of non-peroxide oxygen, sulfur, and N(Y) wherein Y is absent or is H, O, (C₁-C₈)alkyl, phenyl or benzyl, as well as a radical of an ortho-fused bicyclic heterocycle of about eight to ten ring atoms derived therefrom, particularly a benzyl derivative or one derived by fusing a propylene, trimethylene, or tetramethylene diradical thereto.

Heteroaryl encompasses a monocyclic aromatic ring having five or six ring atoms consisting of carbon and one, two, three, or four heteroatoms each selected from the group consisting of non-peroxide oxygen, sulfur, and N(X) wherein X is absent, is H, O, (C₁-C₄)alkyl, phenyl or benzyl, or is a substituent defined elsewhere. Heteroaryl also encompasses a radical of an ortho-fused bicyclic heterocycle of eight to ten ring atoms, particularly a benzyl-derivative or one derived by fusing a propylene, trimethylene, or tetramethylene diradical thereto. Only one ring of the bicyclic heteroaryl need be aromatic.

As used herein, “heterocycle” generally represents a non aromatic heterocyclic group, having from three to about ten ring atoms, which can be saturated or partially unsaturated, containing at least one heteroatom (e.g., one, two, or three) selected from the group consisting of oxygen, nitrogen, and sulfur. Specific, “heterocycle” groups include monocyclic, bicyclic, or tricyclic groups containing one or more heteroatoms selected from the group consisting of oxygen, nitrogen, and sulfur. A “heterocycle” group also can include one or more oxo groups (═O) attached to a ring atom. Non-limiting examples of heterocycle groups include 1,3-dioxolane, 1,4-dioxane, 1,4-dithiane, 2H-pyran, 2-pyrazoline, 4H-pyran, chromanyl, imidazolidinyl, imidazolinyl, indolinyl, isochromanyl, isoindolinyl, morpholine, piperazinyl, piperidine, piperidyl, pyrazolidine, pyrazolidinyl, pyrazolinyl, pyrrolidine, pyrroline, quinuelidine, thiomorpholine, and the like.

As used herein, “carbocyclic biaryl” refers to ortho-fused bicyclic moieties, typically containing ten carbon atoms. An example is naphthalene. The term heterocyclic biaryl as used herein refers to ortho-fused bicyclic moieties containing one, two, three, or four heteroatoms. Examples include indoles, isoindoles, quinolines, isoquinolines, benzofurans, isobenzofurans, benzothiophenes, benzo[c]thiophenes, benzimidazoles, purines, indazoles, benzoxazole, benzisoxazole, benzothiazole, quinoxalines, quinazolines, cinnolines, and the like.

The point of attachment of either the carbocyclic or heterocyclic biaryl can be to any ring atom permitted by the valency of that atom.

Embodiments listed below for radicals, substituents, and ranges, are for illustration only; they do not exclude other defined values or other values within defined ranges for the radicals and substituents.

Carbon chains and their optionally substituted counterparts can be in any branched chain form permitted by the valences and steric requirements of the atoms. Specifically, (C₁-C₈)alkyl can be methyl, ethyl, propyl, isopropyl, butyl, iso-butyl, sec-butyl, tert-butyl, pentyl, 3-pentyl, neopentyl, hexyl, heptyl, octyl, and the like, in any branched chain form.

As used herein, the term “cycloalkyl” encompasses bicycloalkyl (norbornyl, 2.2.2-bicyclooctyl, etc.) and tricycloalkyl (adamantyl, etc.), optionally including 1-2 N, O or S. Cycloalkyl also encompasses (cycloalkyl)alkyl. Thus, (C₃-C₆)cycloalkyl can be cyclopropyl, cyclobutyl, cyclopentyl, cyclohexyl and the like. (C₁-C₈)alkoxy can be methoxy, ethoxy, propoxy, isopropoxy, butoxy, iso-butoxy, sec-butoxy, pentoxy, 3-pentoxy, or hexyloxy, in any branched chain form.

As used herein, (C₂-C₆)alkenyl can be vinyl, allyl, 1-propenyl, 2-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, or 5-hexenyl; (C₂-C₆)alkynyl can be ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, or 5-hexynyl.

As used herein, (C₁-C₆)alkanoyl can be acetyl, propanoyl or butanoyl; halo(C₁-C₆)alkyl can be iodomethyl, bromomethyl, chloromethyl, fluoromethyl, trifluoromethyl, 2-chloroethyl, 2-fluoroethyl, 2,2,2-trifluoroethyl, or pentafluoroethyl; hydroxy(C₁-C₆)alkyl can be hydroxymethyl, 1-hydroxyethyl, 2-hydroxyethyl, 1-hydroxypropyl, 2-hydroxypropyl, 3-hydroxypropyl, 1-hydroxybutyl, 4-hydroxybutyl, 1-hydroxypentyl, 5-hydroxypentyl, 1-hydroxyhexyl, or 6-hydroxyhexyl.

As used herein, (C₁-C₆)alkoxycarbonyl (CO₂R²) can be methoxycarbonyl, ethoxycarbonyl, propoxycarbonyl, isopropoxycarbonyl, butoxycarbonyl, pentoxycarbonyl, or hexyloxycarbonyl.

As used herein, (C₁-C₆)alkylthio can be methylthio, ethylthio, propylthio, isopropylthio, butylthio, isobutylthio, pentylthio, or hexylthio.

As used herein, (C₂-C₆)alkanoyloxy can be acetoxy, propanoyloxy, butanoyloxy, isobutanoyloxy, pentanoyloxy, or hexanoyloxy; aryl can be phenyl, indenyl, or naphthyl; and heteroaryl can be furyl, imidazolyl, triazolyl, triazinyl, oxazoyl, isoxazoyl, thiazolyl, isothiazoyl, pyraxolyl, pyrrolyl, pyrazinyl, tetrazolyl, puridyl (or its N-oxide), thienyl, pyrimidinyl (or its N-oxide), indolyl, isoquinolyl (or its N-oxide) or quinolyl (or its N-oxide).

As used herein, “alkylene” refers to a divalent straight or branched hydrocarbon chain (e.g. ethylene —CH₂CH₂—).

As used herein, “aryl(C₁-C₈)alkylene” for example includes benzyl, phenethyl, 3-phenylpropyl, naphthylmethyl and the like.

As used herein, “treating” or “treatment” covers the treatment of a disease-state in a mammal, and includes: (a) preventing the disease-state from occurring in a mammal, in particular, when such mammal is predisposed to the disease-state but has not yet been diagnosed as having it; (b) inhibiting the disease-state, e.g., arresting or slowing its development; and/or (c) relieving the disease-state, e.g., causing regression of the disease state until a desired endpoint is reached. Treating also includes the amelioration of a symptom of a disease (e.g., lessen the pain or discomfort), wherein such amelioration may or may not be directly affecting the disease (e.g., cause, transmission, expression, etc.).

As used herein, “effective amount” or “therapeutically effective amount” refers to a sufficient amount of the active agent or A_{2A} agonist being administered that would be expected to relieve to some extent one or more of the symptoms of the disease or condition being treated. For example, the result of administration of an A_{2A} adenosine receptor agonist disclosed herein is reduction and/or alleviation of the signs, symptoms, or causes of neuroflammatory diseases. For example, an “effective amount” for therapeutic uses is the amount of A_{2A} adenosine receptor agonist, including a composition as disclosed herein required to provide a decrease or amelioration in disease symptoms without undue adverse side effects. The term “therapeutically effective amount” includes, for example, a prophylactically effective amount. An “effective amount” of an A_{2A} adenosine receptor agonist disclosed herein is an amount effective to achieve a desired pharmacologic effect or therapeutic improvement without undue adverse side effects. It is understood that “an effective amount” or “a therapeutically effective amount” varies, in some embodiments, from patient to patient, due to variation in metabolism of the compound administered, age, weight, general condition of the patient, the condition being treated, the severity of the condition being treated, and the judgment of a prescribing or treating physician and/or other factors which may influence the amount of a medication such as an A_{2A} agonist that is effective. It is also understood that “an effective amount” in one dosing or administration format may differ from “an effective amount” in another dosing or administration format based upon pharmacokinetic, pharmacodynamic, or other considerations. For example, an effective amount for intrathecal administration is not necessarily the same as an effective amount for parenteral administration.

The carbon atom content of various hydrocarbon containing moieties is indicated by a prefix designating the minimum and maximum number of carbon atoms in the moiety, i.e., the prefix Cᵢ-Cⱼ indicates a moiety of the integer “i” to the integer “j” carbon atoms, inclusive. Thus, for example, (C₁-C₈)alkyl refers to alkyl of one to eight carbon atoms, inclusive.

The compounds useful for the methods and composition provided herein are generally named according to the IUPAC or CAS nomenclature system. Abbreviations which are well known to one of ordinary skill in the art may be used (e.g., “Ph” for phenyl, “Me” for methyl, “Et” for ethyl, “cPr” for cyclopropyl, “h” for hour or hours and “rt” for room temperature).

The compounds described herein may have more than one chiral center and may be isolated in optically active and racemic forms. The riboside moiety may be derived from D-ribose. Some compounds may exhibit polymorphism. The compounds useful for the methods and compositions described herein encompass any racemic, optically-active, polymorphic, co-crystalline, or stereoisomeric form, or mixtures thereof, of a compound useful herein, which possess the useful properties described herein, it being well known in the art how to prepare optically active forms (for example, by resolution of the racemic form by recrystallization techniques, or enzymatic techniques, by synthesis from optically-active starting materials, by chiral synthesis, or by chromatographic separation using a chiral stationary phase) and how to determine adenosine agonist activity using the tests described herein, or using other similar tests which are well known in the art.

In cases where compounds are sufficiently basic or acidic to form stable nontoxic acid or base salts, administration of the compounds as salts may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

The compounds useful for the methods and compositions provided herein can be formulated as pharmaceutical compositions and administered to a mammalian host, such as a human patient, in a variety of forms adapted to the chosen route of administration, such as intrathecally.

The pharmaceutical compositions further include: a pharmaceutically acceptable excipient (e.g., carrier) suitable for the route of administration (e.g., intrathecal).

The active compound may be administered intravenously, intra-arterially, intraperitoneally, or intrathecally by infusion or injection. In an embodiment the drug may be administered intradermally, epicutaneously/transdermally, subcutaneously, nasally, intramuscularly, intracardiacally, intraosseously, intravesically, intravitreally, intracavernously, intravaginally, intrauterinely, transdermally, transmucosally, parentally, orally, rectally, sublingually, sublabially, topically, enterally, gastrostomically, duodenally, epidurally, intracerebrally, intracerebroventricularly, intracisternally and/or via inhalation, enema, eye drops or ear drops, as not all embodiments of the present invention are intended to be limited in this respect. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations may contain a preservative to prevent the growth of microorganisms.

The pharmaceutical dosage forms suitable for injection or infusion can include sterile aqueous solutions or dispersions or sterile powders including the active ingredient which are adapted for the extemporaneous preparation of sterile injectable or infusible solutions or dispersions, optionally encapsulated in liposomes. The ultimate dosage form may be sterile, fluid and stable under the conditions of manufacture and storage. The liquid carrier or vehicle can be a solvent or liquid dispersion medium including, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In an embodiment, isotonic agents, for example, sugars, buffers or sodium chloride are included. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various other ingredients as enumerated above, as required, followed by filter sterilization. In the case of sterile powders for the preparation of sterile injectable solutions, the methods of preparation include vacuum drying and the freeze drying techniques, which yield a powder of the active ingredient plus any additional desired ingredient present in the previously sterile-filtered solutions.

Useful dosages of the compounds for the methods and compositions provided herein can be determined by comparing their in vitro activity and in vivo activity in animal models. Methods for the extrapolation of effective dosages in mice, and other animals, to humans are known to the art; for example, see U.S. Pat. No. 4,938,949. Useful dosages of Type IV PDE inhibitors are known to the art; for example, see, U.S. Pat. No. 5,877,180, Col. 12.

The amount of the compound, or an active salt or derivative thereof, required for use in treatment may vary not only with the particular salt selected but also with the route of administration, the nature of the condition being treated and the age and condition of the patient and may be determined at the discretion of the attendant physician, clinician, or healthcare provider. For intrathecal administration, the volume of the composition administered and the concentration of the active ingredient in the composition may be considered when determining a suitable dosage.

The ability of a given compound herein to act as an A_{2A} agonist may be determined using pharmacological models that are well known to the art.

The methods and compositions described herein will be further described by reference to the following detailed examples, which are given for illustration purposes only, and are not intended to be limiting thereof.

### EXAMPLES

A_{2A} agonists useful in methods and compositions provided herein can be prepared as shown in the patents and publications described herein (e.g., U.S. Pat. No. 4,968,697; U.S. Pat. No. 4,956,345; U.S. Pat. No. 5,140,015; U.S. Pat. No. 5,278,150; U.S. Pat. No. 5,593,975; U.S. Pat. No. 6,232,297; U.S. Pat. No. 6,403,567; U.S. Pat. No. 6,642,210; U.S. Pat. No. 7,214,665; U.S. Pat. No. 7,589,076; U.S. Patent Application No. 2006/0040889; U.S. Patent Application No. 2007/0270373). A_{2A} agonists, such as regadenoson (CVT-3146; Lexiscan™), binodenoson (MRE-0470; MRE-0094), CGS-21680, are known in the art and may be used in the present invention. Furthermore, assays to determine whether or not an agent functions as an A_{2A} agonist are well known in the art (e.g., see the above list of patents and publications).

### Example 1

### Experimental Autoimmune Encephalitis (EAE)

Male Dark Agouti rats (225-300 g) were injected, under isoflurane anesthesia, intradermally at the base of the tail with 8.25 μg of myelin oligodendrocyte glycoprotein (MOG injection) (suspension made in incomplete Freund's adjuvant). Rats were monitored daily for body weight changes and motor symptoms. At the onset of the first motor symptoms (e.g., tail paralysis, approximately 10-15 days after MOG injection), the A_{2A} agonist (e.g., 10 μM CGS21680, either 1 or 10 μM ATL313) or vehicle (0.01% DMSO in sterile saline) were administered intrathecally as a single bolus, under brief isoflurane anesthesia. Motor score and body mass were assessed daily. On day 24 after the onset of motor symptoms, or before if motor score was 5 or higher, tissue was collected and processed for immunohistochemical analysis of the microglial activation marker, OX-42, the macrophage activation marker, cd68 and the alternative activation marker, cd163.

### Example 2

### Motor Symptoms with CGS21680

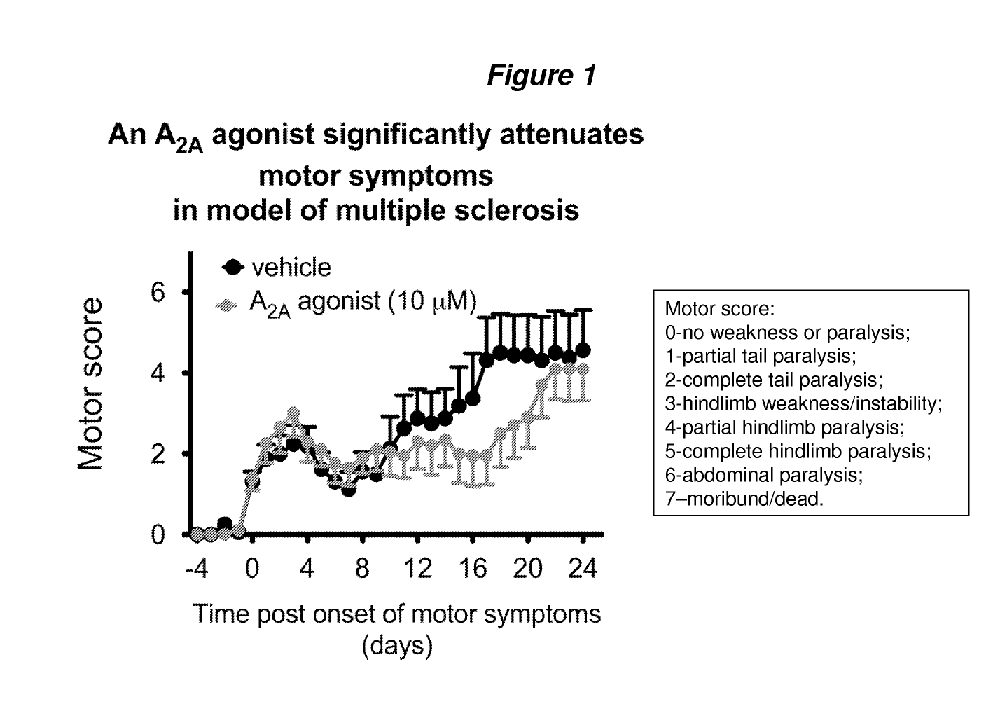

The EAE model described above was used. Motor score was assessed three days before and daily post MOG administration (8.25 ug intradermally). At the onset of motor symptoms (partial tail paralysis, motor score 1) rats were randomly injected with an A_{2A} agonist, CGS21680 (3-[4-[2-[[6-amino-9-[(2R,3R,4S,5S)-5-(ethylcarbamoyl)-3,4-dihydroxy-oxolan-2-yl]purin-2-yl]amino]ethyl]phenyl]propanoic acid) (10 μM) or vehicle. CGS21680 reduced the severity of motor symptoms. The results of this study are shown in FIG. 1, which uses the following scoring paradigm.
Motor score: 0—no weakness or paralysis
Motor score: 1—partial tail paralysis
Motor score: 2—complete tail paralysis
Motor score: 3—hindlimb weakness/instability
Motor score: 4—partial hindlimb paralysis
Motor score: 5—complete hindlimb paralysis
Motor score: 6—abdominal paralysis
Motor score: 7—moribund/dead

A single intrathecal injection of an A_{2A} agonist, CGS21680, attenuated motor symptoms associated with EAE, a model of relapsing remitting multiple sclerosis

### Example 3

### Survival Curve with CGS21680

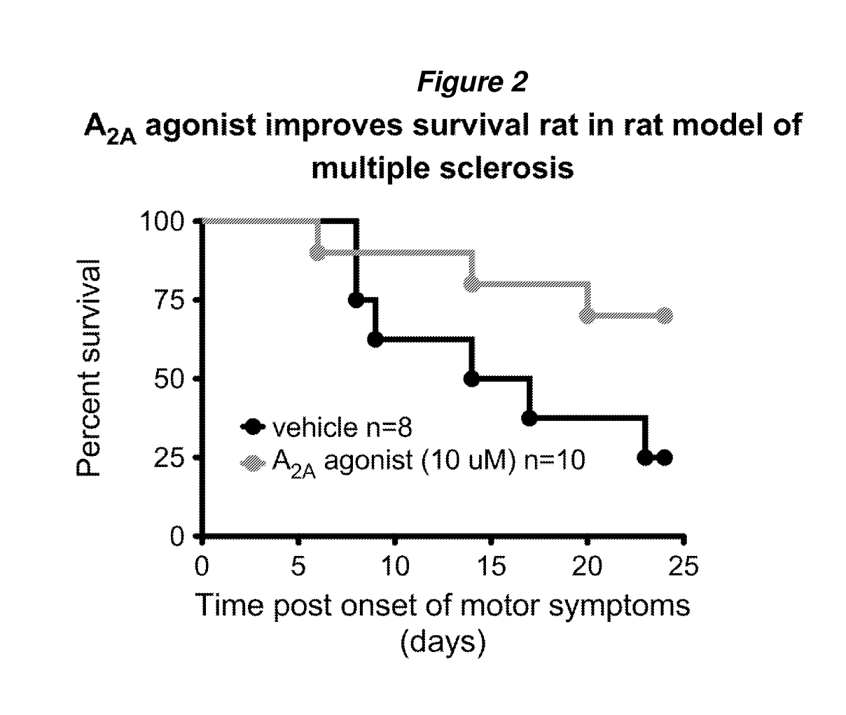

In a separate group of rats from Example 1, MOG was intradermally administered as described above. At the onset of motor symptoms (partial tail paralysis, motor score 1) rats were randomly injected with an A_{2A} agonist, CGS21680 (10 μM), or vehicle. The time at which motor score reached 6 or higher after the onset of motor symptoms is represented on the survival curve shown in FIG. 2. On day 25 there was a significant difference in percentage of survival after CGS21680 compared to vehicle injected rats (P<0.05). A single intrathecal injection of an A_{2A} agonist, CGS21680, improved survival percentage in rats with EAE.

### Example 4

### Immunohistochemistry of the Dorsal Spinal Cord

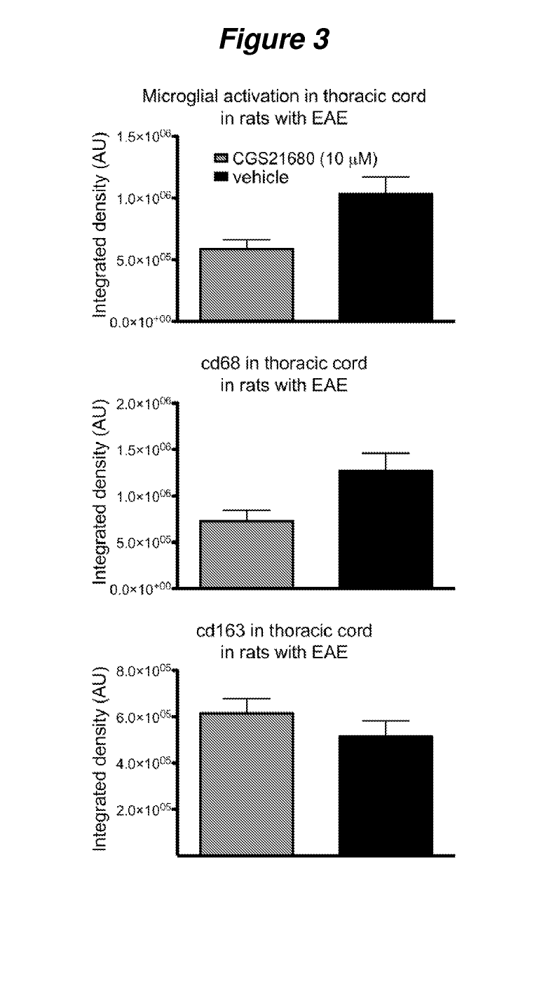

At the time at which rats deteriorated to a motor score of 6 or higher, or 24 days post onset of motor symptoms, spinal cord tissue was collected and processed for immunohistochemical analysis of OX-42 (microglial activation marker), cd68 (classically activated macrophage marker) and cd163 (an alternatively activated macrophage marker). As shown in FIG. 3, there was a significant reduction in OX-42 and cd68 staining, but not cd163, in the rats that received CGS21680 compared to vehicle (P<0.05). The improvement in motor symptoms and survival rate following intrathecal CGS21680 administration in EAE rats is correlated to an attenuation of activated macrophages/microglia within the spinal cord (FIG. 3).

### Example 5

### Motor Symptoms with ATL313

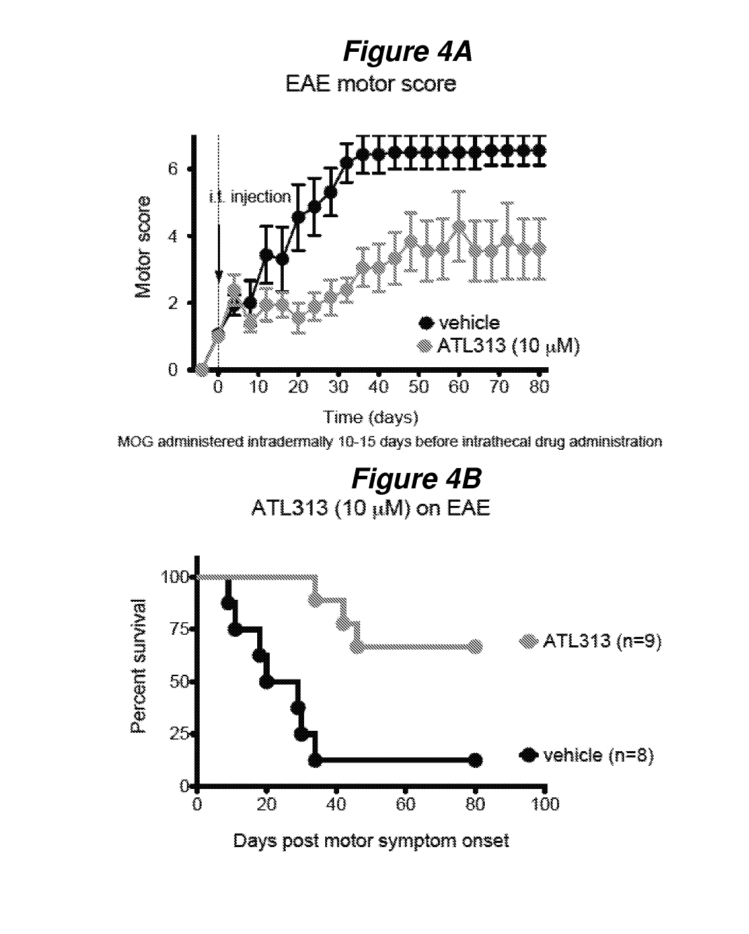

ATL313 (4-{3-[6-amino-9-(5-cyclopropylcarbamoyl-3,4-dihydroxy-tetrahydro-furan-2-yl)-9H-purin-2-yl]-prop-2-ynyl}-piperidine-1-carboxylic acid methyl ester) (single bolus 1 μM-not shown and 10 μM) was used in the EAE model as described above (8-9 rats/group). The change in motor symptoms and the survival curve for ATL313 (10 μM) are shown in FIGS. 4A and 4B, respectively. 1 μM ATL313 produced a reduction in motor score lasting approximately 10 days. However, a single intrathecal injection of 10 μM ATL313 arrested the progression of motor score symptoms for >80 days and increased survival rate in rats injected with ATL313 (MOG+ATL313) compared to rats injected with vehicle (MOG+vehicle). As seen in FIG. 4B, only ⅛ positive control (MOG+vehicle) rats survived by 35 days, whereas 6/9 MOG+ATL313 rats were still alive at 70 days and with stabilized motor scores (FIG. 4A). A single intrathecal injection of an A_{2A} agonist, ATL313, attenuated motor symptoms associated with EAE and improved survival percentage in rats with EAE. Fluid-attenuated inversion recovery (FLAIR) and T2-weighted MRI on rat spines also showed well-defined hyperintensity area in animal with low motor score, while only minimal abnormalities were seen in ATL313 treated animals with preserved motor scores.

### Example 6

### Sub-Analysis of “Responders”

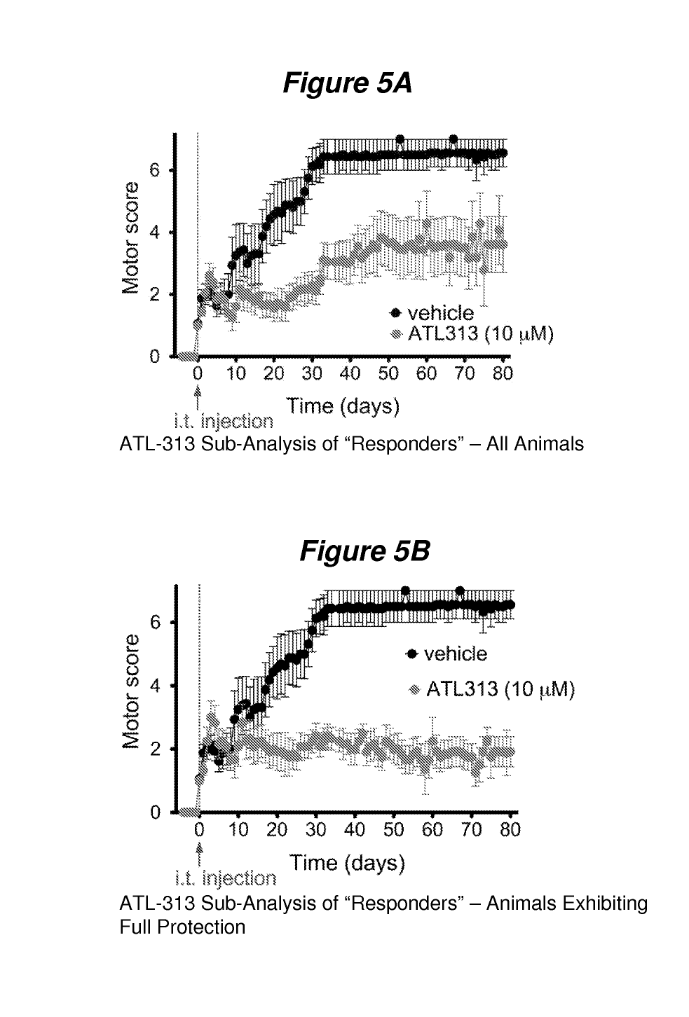

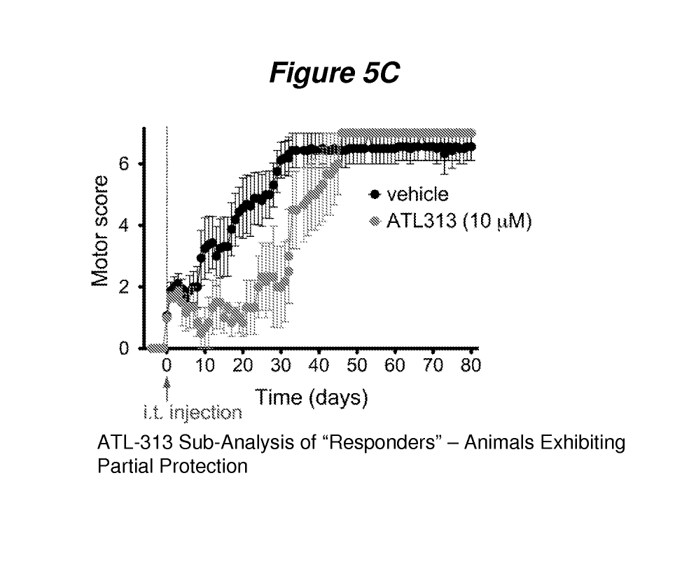

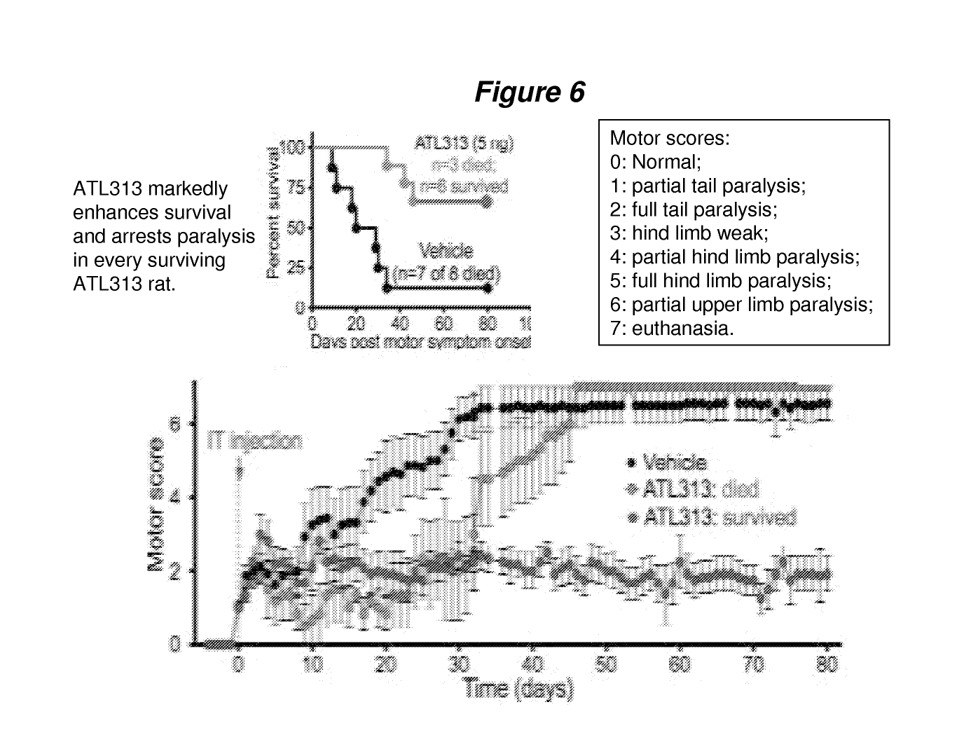

5 ng of ATL313 was administered intrathecally into the spinal cord of Male Dark Agouti rats as described above, resulting in good attenuation of progression of motor impairment/dysfunction induced by EAE. ATL313 was administered as a single bolus at the time of initial onset of motor impairment (when mild paralysis of the tail was observed). As can be seen in FIG. 5A (all the animals included), the variability of the motor responses appeared to increase around ˜35 days after drug administration. That duration is about the same duration of effect as identified in both the spinal nerve ligation and the chronic constriction model where ATL313 attenuates neuropathic pain. Investigating the individual rat responses more closely, a bimodal distribution of response was identified after ˜35 day after injection. FIG. 5B shows the first group of animals where the motor deterioration is arrested (n=6) with no further progression of motor impairment. FIG. 5C (n=3) shows the second group of rats, where the motor symptoms at around day 35 continued on the usual course of motor dysfunction. Therefore, the results suggest that, at worst, the motor effects of EAE are attenuated for about ˜35 days and that possibly a second injection at that time, as identified in the model of CCI for neuropathic pain, would continue to arrest progression of the motor dysfunction or, as seen FIG. 5B, a complete arrest of motor symptoms with no further development of impairment may also occur.

### Example 7

### Clinical Trials

The A_{2A} agonist, ATL313, is intrathecally administered to 20 patients suffering from multiple sclerosis and showing clinical symptoms. Symptoms are described and rated by the patient prior to treatment with ATL313. ATL313 is given at a dose of 1 to 15 μg/kg in a single bolus injection. Symptoms are monitored and rated by the patients every 1-2 hours following treatment for the first 12 hours and then daily for up to 4 weeks. If symptoms remain, a second intrathecal administration with ATL313 is given. Symptoms are monitored and rated by the patients every 1-2 hours following treatment for the first 12 hours and then daily for up to 4 weeks.

### Example 8

### Characterization of the Suppression of EAE Induced Paralysis, Sensory Disturbances, & Death by Intrathecal (IT) Versus Systemic A2A Receptor Agonist (ATL313)

ATL313 is tested at 0, 10 and 100 μM. Before MOG treatment, rats are habituated to the behavioral testing procedure for four consecutive days and baseline measurements are recorded from calibrated mechanical stimuli (von Frey filaments). Rats are then injected intradermally into the base of the tail, under brief isoflurane anesthesia, with Myelin Oligodendrocyte Glycoprotein (MOG; 8.25 μg) or vehicle (incomplete Freund's adjuvant) as described in Sloane, E., et al., 2009. Anti-inflammatory cytokine gene therapy decreases sensory and motor dysfunction in experimental Multiple Sclerosis: MOG-EAE behavioral and anatomical symptom treatment with cytokine gene therapy. Brain Behav Immun. 23, 92100. At the peak of the second cycle of motor paralysis, ATL313 or vehicle is administered intrathecally, under brief isoflurane anesthesia. After MOG injection, von Frey testing will occur every 2-3 days (to prevent behavioral sensitization on this test, which would confound results) while motor score & body weight will be measured daily. The study is terminated before the onset of respiratory symptoms. The time course will be followed out to assess the duration of each drug at each dose.

Pilot studies are conducted to determine an optimal dose & dosing. Dose escalation is initiated with a single administration of 1 μg/kg administered i.p. The study will follow the same experimental design as that described above except that systemic dosing (optimal dose determined by pilot studies) are administered upon initial clinical signs and in a separate group of rats, at the height of the second peak of paralysis. A group of non-MOG injected rats will be included to assess the effect of systemic drug administration, in the absence of inflammation.

A third study will proceed similarly to those described above except that 2 groups of rats will receive active treatment (ATL313) and 1 group will receive vehicle, at the onset of first motor paralysis. The most successful dose will be selected assuming that a comparable effect is found whether the drug is administered at the first sign of motor impairment compared to at the time of the second peak. Once the rats receiving vehicle have reached terminal motor impairment, a second injection of MOG will be administered to the rats receiving ATL313. The rats will then receive either vehicle or ATL313 (same dose as the first injection) once motor function shows sign of deterioration.

A fourth study will proceed similarly to those above except that all rats will receive active treatment (ATL313) either intrathecally or systemically, at the onset of first motor paralysis. The most successful route of administration will then be selected. Once the drug treatment attains maximal efficacy, rats receive once daily intrathecal (IT) injections of either neutralizing anti-IL-10 IgG or normal IgG as described in Loram, L. C., et al., 2009. Enduring reversal of neuropathic pain by a single intrathecal injection of adenosine 2A receptor agonists: a novel therapy for neuropathic pain. J. Neurosci. 29 14015-25, and also described in Sloane, E., et al., 2009. Immunological priming potentiates non-viral anti-inflammatory gene therapy treatment of neuropathic pain. Gene Ther. IgG treatments are continued until it is clear whether or not blockade of IL-1 0 affects symptomatology. A multi-day treatment is warranted so to avoid false negatives. This study is preceded by pilot studies so to define the optimal study design. If pilot studies indicate that continuing observations beyond termination of anti-IL-10 IgG dosing will prove insightful (i.e., to indicate recovery of drug efficacy once anti-IL-10 IgG is cleared), this will then be pursued. If differences in effects between drug administration at the onset of motor paralysis or the peak of the second cycle, the study will be duplicated using anti-IL-10 IgG or IgG following the administration of ATL313 at the peak of the second cycle. If it is found that systemic administration of ATL313 is more successful than intrathecal administration, then pilot studies will be run to identify whether a different response if obtained by systemic or intrathecal anti-IL-10. If differences are obtained both options will be explored.

### Example 9

### Characterization of the Impact of an A_{2A} Receptor Agonist (ATL313) on CNS Pathology Induced by EAE

All tissues (four percent glutaraldehyde fixed) will first be imaged using the MRI results as guidance, for EM analyses. A separate group of rats is required for the IHC as glutaraldehyde fixative detrimentally affects IHC analyses.

Three of the four groups of rats will be injected intradermally with MOG as above. At the peak of the second expression of motor symptoms, group one (EAE positive control; n=10) will be injected intrathecally with the appropriate vehicle. Tissues are collected for this group upon euthanasia when the paralysis extends to the forelimbs. Groups two and three (n=10/group) will be injected intrathecally, at the peak of the second expression of motor symptoms. Tissues from group two (EAE-therapeutic, matched tissue collection times) will be collected at the identical points in time when group one rats are euthanized (averaging around day 22). Tissues from group three (EAE-therapeutic, extended time point) will be collected as late in time as motor symptoms remain stably suppressed. When tissues are collected from rats in group three, tissues from time matched rats from group four (naive controls) will be collected as well.

It has been shown that in the experimental models of MS, including EAE, demyelinating lesions with extensive inflammation result in hypelintense areas on T2-weighted images, namely T2-MRI (anatomical lesion localization & volume calculations; hyperintensive area in white matter correspond to inflammation & demyelination), T2-MRI mapping (high T2 values in the grey matter represent tissue edema; high T2 values in the white matter correlate with necrosis), DW-MRI (low ADC-values reflect the white matter lesions & demyelination; in the grey matter, they are reflective for edema), Magnetization Transfer (T1-based) MRI (low MT ratios (MTR) are a well-known marker for demyelination). Chemically-fixed nervous tissues are well-suited for high-resolution, time-intensive MRI acquisition without motion artifacts. Four percent glutaraldehyde fixative solution is used to preserve spinal cord and brain since it causes only minimal T2 reduction ex vivo. Briefly, control animals (n=6-8) as well as EAE animals (with and without treatment, each n=6-8) will be sacrificed. The spinal cord and brain tissues will be preserved in four percent glutaraldehyde. All specimens undergo MRI using a Bruker 4.7 Tesla/16 cm animal scanner. Two T2-weighted MRI sequences will be used, RARE (rapid acquisition with relaxation enhancement: field of view 6.4 cm; TR/TE=2500/80 ms) and FLAIR (fluid attenuation inverse recovery: field of view 6.4 cm; TRITE=8900/80 ms) will be used to detect hyperintense lesions, calculate signal intensities and T2-relaxation times. Each spinal cord will be acquired with four bed positions (to scan the entire cord using 6.4-cm sections) and each brain will require one bed position. The prolonged T2 relaxation times and extensive hyperintense area will correlate with motor scores (Watkins) and immunohistochemical analysis (Macklin). If T2- and signal intensity standard deviations among each group are too high (which is possible when comparing ex vivo specimens) the number of animals per group will be increased.

Upon completion of MR imaging, the intact brain & spinal cord of each rat will be dissected out. A separate group of rats perfused with four percent paraformaldehyde will be used for the immunohistochemistry studies. Brains & spinal cords of each rat will be postfixed overnight in four percent paraformaldehyde and stored in cryoprotection medium until microtome sectioning. Floating sections (30 mm) will be analyzed as in earlier studies. Transverse sections of spinal cord will be analyzed for immune cell changes, myelin loss, remyelination and apoptosis. Brain sections will be analyzed as needed.

The extent of immune cell infiltration & the phenotype of the immune cells will be assessed in a separate squad of rats, staining with H&E to detect inflammatory infiltrates and with antibodies for CD4, CD8, Arg1, cd11b and cd163 using ABC Vectastain. Sections will also be stained for amyloid precursor protein (APP, for axonal pathology). Integrated densitometry will be analyzed using Image J (NIH freeware). For the measurement of apoptosis, sections will be stained with the Apoptag detection kit, & the numbers of apoptotic cells will be quantified manually & reported as positive cells/mm.

In order to assess the extent of demyelination, semithin sections of the cervical, thoracic & lumbar spinal cord will be taken (10 sections/rat). Sections will be osmicated, dehydrated & embedded. One micron thick sections will be cut & stained with toluidine blue for myelin. The relative extent of myelin loss in sections will be determined by demarcating the demyelinated area in toluidine blue-stained serial sections by light microscopy. If the MR studies demonstrate brain lesions, sections from the brain will also be analyzed.

To measure axonal de/remyelination, ultrathin sections (85 nm) will be cut & stained with uranyl acetate & lead citrate. EMs will be taken using FEI Technai TEM at 80 kv or Philips CM10 TEM at 80 kv. The g ratio & axon diameter will be determined by the diameters of axons and the corresponding myelinated fibers calculated from their areas, derived using Scion Image. Only relatively circular, well fixed & well defined axons will be used for measurement. At least one hundred randomly selected axons will be analyzed per animal per region. The total number of axons will be quantified for a given volume of the spinal cord. This will provide a measure of axonal loss in these animals. Totally demyelinated axons will be quantified & the g ratio of the myelinated axons will be analyzed. An increased g ratio (thinner myelin) will indicate remyelinated axons.

### Example 10

### Characterization of the Impact of an A₂ₐ Receptor Agonist (ATL313) on the Proinflammatory Vs. Anti-Inflammatory State of Spinal Cord Immune Cells in EAE

Studies will be completed in order to indentify whether the “foamy” macrophages present in spinal cord EAE lesions that have previously been shown to exhibit an alternatively activated phenotype, are more prevalent following A_{2A} agonist administration and have increased expression of known alternative activation markers. The optimal dose of ATL313 will be tested in EAE rats against vehicle controls and naive rats. Four cm of spinal cord tissue from the bottom of the lumbar enlargement upwards will be collected in vehicle rats at the time of their second peak of relapse. The meninges surrounding the section of spinal cords, containing meningeal macrophages, will be carefully removed and assayed separately for gene expression (IL-10 for anti-inflammatory cytokine, IL-10 for pro-inflammatory cytokine, MHCII and cd11b, markers of classically activated macrophages/microglia, Arg1, YMI and CCL18, markers of alternatively activated macrophages/microglia). 10 genes may be measured via mRNA analysis from each individual tissue sample. The spinal cord will be placed in a 100 mm Petri dish & finely minced. The tissue will be enzymatically dissociated, serially triturated & then filtered through 0.4 μm filter. The homogenate will then be immunopanned with positive selection such that the cells attached to the plate are the macrophages of interest. The isolated cells will be divided into 2 tubes: PCR analysis (IL-10, IL-1, MHCII, cd1 1b, Arg1, YM1 and CCL18) and western blot analysis (IL-10, IL-1 and Arg). A small sample of the cells will be plated on glass cover slips & stained with oil red 0 for detection of myelin degradation byproducts. Spinal cords from ten rats per group will be isolated & processed for macrophage analysis.

All patents, patent applications, and published references cited herein are hereby incorporated by reference in their entirety. It will be appreciated that several of the above-disclosed and other features and functions, or alternatives thereof, may be desirably combined into many other different systems or applications. Various presently unforeseen or unanticipated alternatives, modifications, variations, or improvements therein may be subsequently made by those skilled in the art which are also intended to be encompassed by the embodiments described herein.

Any of the above dependent claims may be combined with each other. Although the claims are listed as singly dependent claims, the dependent claims may be multiply dependent upon any of the other claims. Some embodiments may be claimed by a claim that combines any independent claim with another independent claim.

1. A method of treating or ameliorating a neuroinflammatory disease or a symptom thereof in a patient in need thereof, wherein the method comprises administering to the patient a therapeutically effective amount of an A_{2A} agonist,
wherein treating or ameliorating the neuroinflammatory disease or the symptom thereof comprises treating or reducing pain, improving or stabilizing motor function, increasing or improving survival rate, increasing remyelination, decreasing or preventing demyelination, sparing neuronal death, suppressing motor paralysis, or any combinations thereof.

2-9. (canceled)

10. The method of claim 1,
wherein IL-10 production is increased in the patient.

11. The method of claim 1,
wherein IL-10 levels are increased in the patient.

12. The method of claim 1, wherein the volume administered to the patient is in the range of 0.2-10 μL.

13. The method of claim 1, wherein the amount of A_{2A} agonist administered to the patient ranges from 0.1 to 500 ng per administration.

14. The method of claim 1, wherein the A_{2A} agonist is administered as part of a pharmaceutical composition, the composition further including a pharmaceutically acceptable excipient.

15. A pharmaceutical composition comprising an A_{2A} agonist, wherein the composition is formulated for intrathecal administration, wherein the concentration of the A_{2A} agonist in the composition ranges from 1-100 μM.

16. The method of claim 1, wherein the A_{2A} agonist comprises a substituted 6-amino-9-(tetrahydrofuran-2′-yl)purine.

17. The method of claim 16, wherein the A_{2A} agonist comprises a 6-amino-9-(3′,4′-dihydroxy-tetrahydrofuran-2′-yl)purine compound substituted at the 3- and 5′-positions, or a pharmaceutically acceptable salt thereof.

18. The method of claim 17, wherein the A2A agonist comprises:
a 5-[6-amino-2-(3-piperidin-4-yl-prop-1-ynyl)-purin-9-yl]-3,4-dihydroxy-tetrahydrofuran-2-carboxylic acid cyclopropylamide compound, substituted on the piperidine nitrogen,
a 4-{3-[6-amino-9-(5-cyclopropylcarbamoyl-3,4-dihydroxy-tetrahydro-furan-2-yl)-9H-purin-2-yl]-prop-2-ynyl}-piperidine-1-carboxylic acid ester:
a 5-[6-amino-2-(3-piperidin-4-yl-prop-1-ynyl)-purin-9-yl]-3,4-dihydroxy-tetrahydrofuran-2-carboxylic acid ethylamide compound, substituted on the piperidine nitrogen,
a 4-{3-[6-amino-9-(5-ethylcarbamoyl-3,4-dihydroxy-tetrahydro-furan-2-yl)-9H-purin-2-yl]-prop-2-ynyl}-piperidine-1-carboxylic acid ester;
or a pharmaceutically acceptable salt thereof.

19-21. (canceled)

22. The method of claim 1, wherein the A_{2A} agonist is selected from the group consisting of:
(a) a compound of formula 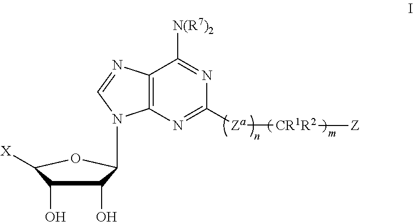
wherein:
Z^{a} is C≡C, O, NH, or NHN═CR^{3a};
n is 0 or 1;
Z is CR³R⁴R⁵ or NR⁴R⁵;
each R¹ is independently hydrogen, halo, —OR^{a}, —SR^{a}, (C₁-C₈)alkyl, cyano, nitro, trifluoromethyl, trifluoromethoxy, (C₃-C₈)cycloalkyl, heterocycle, heterocycle(C₁-C₈)alkylene-, aryl, aryl(C₁-C₈)alkylene-, heteroaryl, heteroaryl(C₁-C₈)alkylene-, —CO₂R^{a}, R^{a}C(═O)O—, R^{a}C(═O)—, —OCO₂R^{a}, R^{b}R^{c}NC(═O)O—, R^{a}OC(═O)N(R^{b})—, R^{b}R^{c}N—, R^{b}R^{c}NC(═O)—, R^{a}C(═O)N(R^{b})—, R^{b}R^{c}NC(═O)N(R^{b})—, R^{b}R^{c}NC(═S)N(R^{b})—, —OPO₃R^{a}, R^{a}OC(═S)—, R^{a}C(═S)—, —SSR^{a}, R^{a}S(═O)—, R^{a}S(═O)₂—, or —N═NR^{b};
each R² is independently hydrogen, halo, (C₁-C₈)alkyl, (C₃-C₈)cycloalkyl, heterocycle, heterocycle(C₁-C₈)alkylene-, aryl, aryl(C₁-C₈)alkylene-, heteroaryl, or heteroaryl(C₁-C₈)alkylene-;
alternatively, R¹ and R² and the atom to which they are attached is C═O, C═S or C═NR^{d},
R⁴ and R⁵ are independently H or (C₁-C₈)alkyl;
alternatively, R⁴ and R⁵ together with the atom to which they are attached form a saturated, partially unsaturated, or aromatic ring that is mono-, bi- or polycyclic and has 3, 4, 5, 6, 7, 8, 9 or 10 ring atoms optionally having 1, 2, 3, or 4 heteroatoms selected from O, S(O)₀₋₂, and amine in the ring;
wherein R⁴ and R⁵ are independently substituted with 0-3 R⁶ groups or any ring including R⁴ and R⁵ is substituted with from 0 to 6 R⁶ groups;
each R⁶ is independently hydrogen, halo, —OR^{a}, —SR^{a}, (C₁-C₈)alkyl, cyano, nitro, trifluoromethyl, trifluoromethoxy, (C₁-C₈)cycloalkyl, (C₆-C₁₂)bicycloalkyl, heterocycle, heterocycle(C₁-C₈)alkylene-, aryl, aryl(C₁-C₈)alkylene-, heteroaryl, heteroaryl(C₁-C₈)alkylene-, —CO₂R^{a}, R^{a}C(—O)O—, R^{a}C(═O)—, —OCO₂R^{a}, R^{b}R^{c}NC(═O)O—, R^{a}OC(═O)N(R^{b})—, R^{b}R^{c}N—, R^{b}R^{c}NC(═O)—, R^{a}C(═O)N(R^{b})—, R^{b}R^{c}NC(═O)N(R^{b})—, R^{b}R^{c}NC(═O—S)N(R^{b})—, —OPO₃R^{a}, R^{a}OC(═S)—, R^{a}C(═S)—, —SSR^{a}, R^{a}S(═O)—, —NNR^{b}, or two R⁶ groups and the atom to which they are attached is C═O, C═S; or two R⁶ groups together with the atom or atoms to which they are attached can form a carbocyclic or heterocyclic ring including from 1-6 carbon atoms and 1, 2, 3, or 4 heteroatoms selected from —O—, S(O)₀₋₂, and amine in the ring;
R³ is hydrogen, halo, —OR^{a}, (C₁-C₈)alkyl, cyano, nitro, trifluoromethyl, trifluoromethoxy, (C₃-C₈)cycloalkyl, heterocycle, heterocycle(C₁-C₈)alkylene-, aryl, aryl(C₁-C₈)alkylene-, heteroaryl, heteroaryl(C₁-C₈)alkylene-, —CO₂R^{a}, R^{a}C(═O)O—, R^{a}C(═O)—, —OCO₂R^{a}, R^{b}R^{a}NC(═O)O—, R^{a}OC(═O)N(R^{b})—, R^{b}RN—, R^{b}R^{c}NC(═O)—, R^{a}C(═O)N(R^{b})—, R^{b}R^{c}NC(═O)N(R^{b})—, R^{b}R^{c}NC(—S)N(R^{b})—, —OPO₃R^{a}, R^{a}C(═S)—, R^{a}C(═S)—, —SSR^{a}, R^{a}S(═O)—, R^{a}S(═O)₂—, —NNR^{b}; or if the ring formed from CR⁴R⁵ is aryl or heteroaryl or partially unsaturated then R³ can be absent;
R^{3a} is hydrogen, (C₁-C₈)alkyl, or aryl;
each R⁷ is independently hydrogen, (C₁-C₈)alkyl, (C₃-C₈)cycloalkyl, aryl, aryl(C₁-C₈)alkylene, heteroaryl, or heteroaryl(C₁-C₈)alkylene-;
X is —CH₂OR^{a}, —CO₂R^{a}, —CH₂OC(O)R^{a}, —C(O)NR^{b}R^{c}, —CH₂SR^{a}, —C(S)OR^{a}, —CH₂OC(S)R^{a}, —C(S)NR^{b}R^{c}, or —CH₂N(R^{b})(R^{c});
alternatively, X is an aromatic ring of the formula: 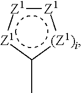
wherein:
each Z¹ is —O—, S(O)₀₋₂, —C(R⁸)—, or —NR⁸—, provided that at least one Z¹ is —O—, S(O)₀₋₂, or —NR⁸—;
each R⁸ is independently hydrogen, (C₁-C₈)alkyl, (C₁-C₈)alkenyl, (C₃-C₈)cycloalkyl, (C₃-C₈)cycloalkyl(C₁-C₈)alkylene, (C₃-C₈)cycloalkenyl, (C₃-C₈)cycloalkenyl(C₁-C₈)alkylene, aryl, aryl(C₁-C₈)alkylene, heteroaryl, or heteroaryl(C₁-C₈)alkylene, wherein any of the alkyl or alkenyl groups of R⁸ are optionally interrupted by —O—, —S—, or —N(R^{a})—;
wherein any of the alkyl, cycloalkyl, heterocycle, aryl, or heteroaryl, groups of R¹, R², R³, R^{3a}, R⁶, R⁷ and R⁸ is optionally substituted on carbon with 1, 2, 3, or 4 substituents selected from the group consisting of halo, —OR^{a}, —SR^{a}, (C₁-C₈)alkyl, cyano, nitro, trifluoromethyl, trifluoromethoxy, (C₃-C₈)cycloalkyl, (C₆-C₁₂) bicycloalkyl, heterocycle, heterocycle(C₁-C₈)alkylene-, aryl, aryloxy, aryl(C₁-C₈)alkylene-, heteroaryl, heteroaryl(C₁-C₈)alkylene-, —CO₂R^{a}, R^{a}C(═O)O—, R^{a}C(═O)—, —OCO₂R^{a}, R^{b}R^{c}NC(═O)O—, R^{a}OC(═O)N(R^{b})—, R^{b}R^{c}N—, R^{b}R^{c}NC(═O)—, R^{a}C(═O)N(R^{b})—, R^{b}R^{c}NC(═O)N(R^{b})—, R^{b}R^{c}NC(═S)N(R^{b})—, —OPO₃R^{a}, R^{a}OC(═S)—, R^{a}C(═S)—, —SSR^{a}, R^{a}S(═O)ₚ—, R^{b}R^{c}NS(O)ₚ—, and —N═NR^{b};
wherein any (C₁-C₈)alkyl, (C₃-C₈)cycloalkyl, (C₆-C₁₂)bicycloalkyl, (C₁-C₈)alkoxy, (C₁-C₈)alkanoyl, (C₁-C₈)alkylene, or heterocycle, is optionally partially unsaturated;
each R^{a}, R^{b} and R^{c} is independently hydrogen, (C₁-C₁₂)alkyl, (C₁-C₈)alkoxy, (C₁-C₈)alkoxy-(C₁-C₁₂)alkylene, (C₃-C₈)cycloalkyl, (C₃-C₈)cycloalkyl-(C₁-C₁₂)alkylene, (C₁-C₈)alkylthio, amino acid, aryl, aryl(C₁-C₈)alkylene, heterocycle, heterocycle-(C₁-C₈)alkylene, heteroaryl, or heteroaryl(C₁-C₈)alkylene;
alternatively R^{b} and R^{c}, together with the nitrogen to which they are attached, form a pyrrolidino, piperidino, morpholino, or thiomorpholino ring;
wherein any of the alkyl, cycloalkyl, heterocycle, aryl, or heteroaryl groups of R^{a}, R^{b} and R^{c} is optionally substituted on carbon with 1 or 2 substituents selected from the group consisting of halo, —(CH₂)ₐOR^{e}, —(CH₂)ₐSR^{e}, (C₁-C₈)alkyl, (CH₂)ₐCN, (CH₂)ₐNO₂, trifluoromethyl, trifluoromethoxy, —(CH₂)ₐCO₂R³, (CH₂)ₐNR^{e}R^{e}, and (CH₂)ₐC(O)NR^{e}R^{e};
R^{d} is hydrogen or (C₁-C₆)alkyl;
R^{e} is independently selected from H and (C₁-C₆)alkyl;
a is 0, 1, or 2;
m is an integer ranging from 0 to 8; and
p is an integer ranging from 0 to 2;
provided that m is at least 1 when Z is NR⁴R⁵;
(b) a compound recited in Table 1;
(c) a compound of formula II: 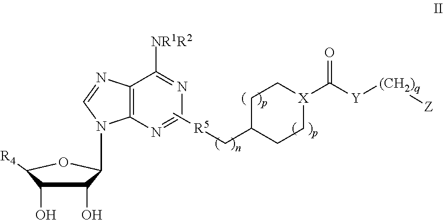
wherein:
R¹ and R² independently are selected from the group consisting of H, (C₁-C₈)alkyl, (C₃-C₈)cycloalkyl, (C₃-C₈)cycloalkyl(C₁-C₈ alkylene, aryl, aryl(C₁-C₈)alkylene, heteroaryl, heteroaryl(C₁-C₈)alkylene-, diaryl(C₁-C₈)alkylene, and diheteroaryl(C₁-C₈)alkylene, wherein the aryl and heteroaryl rings are optionally substituted with 1-4 groups independently selected from fluoro, chloro, iodo, bromo, methyl, trifluoromethyl, and methoxy,
each R independently is selected from the group consisting of H, (C₁-C₄)alkyl, cyclopropyl, cyclobutyl, and (CH₇)ₐcyclopropyl;
X is CH or N, provided that when X is CH then Z cannot be substituted with halogen, (C₁-C₆)alkyl, hydroxyl, amino, or mono- or di-(C₁-C₆-alkyl)amino;
Y is selected from the group consisting of O, NR¹, —(OCH₂CH₇CH₂O)ₘCH₂—, and —(NR¹CH₂CH₂O)ₘCH₂—, provided that when Y is O or NR¹, then at least one substituent is present on Z;
Z is selected from the group consisting of 5-membered heteroaryl, 6-membered aryl, 6-membered heteroaryl, carbocyclic biaryl, and heterocyclic biaryl, wherein the point of attachment of Y to Z is a carbon atom on Z, wherein Z is substituted with 0-4 groups independently selected from the group consisting of F, Cl, Br, I, (C₁-C₄)alkyl, —(CH₂)ₐOR³, —(CH₂)ₐNR³R³, —NHOH, —NR³NR³R³, nitro, —(CH₂)ₐCN, —(CH₂)ₐCO₂R³, —(CH₂)ₐCONR³R³, trifluoromethyl, and trifluoromethoxy;
alternatively, Y and Z together form an indolyl, indolinyl, isoindolinyl, tetrahydroisoquinolinyl, or tetrahydroquinolinyl moiety wherein the point of attachment is via the ring nitrogen and wherein said indolyl, indolinyl, isoindolinyl, tetrahydroisoquinolinyl, or tetrahydroquinolinyl moiety, which is substituted with 0-4 groups independently selected from the group consisting of F, Cl, Br, I, (C₁-C₄)alkyl, —(CH₂)ₐOR³, —(CH₂)ₐNR³R³, —NHOH, —NR³NR³R³, NO₂, —(CH₂)ₐCN, —(CH₂)ₐCO₂R₃, —(CH₂)ₐCONR³R³, CF₃, and OCF₃;
R³ is independently selected from the group consisting of H, (C₁-C₆)alkyl, cycloalkyl, aryl, and heteroaryl;
R⁴ is selected from the group consisting of CH₂OR, C(O)NRR, and CO₂R;
R⁵ is selected from the group consisting of CH₂CH₂, CH═CH, and C═C;
a is selected from 0, 1, and 2;
m is selected from 1, 2, and 3;
n is selected from 0, 1, and 2;
each p independently is selected from 0, 1, and 2; and,
q is selected from 0, 1, and 2;
(d) a compound recited in Table 2;
(e) a compound of formula (Ib)-(Id): 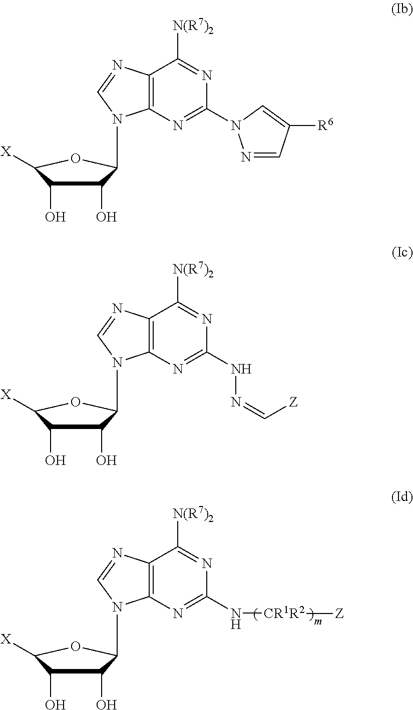
wherein:
each R¹ is independently hydrogen, halo, —OR^{a}, —SRa, (C₁-C₈)alkyl, cyano, nitro, trifluoromethyl, trifluoromethoxy, (C₃-C₈)cycloalkyl, heterocycle, heterocycle(C₁-C₈)alkylene-, aryl, aryl(C₁-C₈)alkylene-, heteroaryl, heteroaryl(C₁-C₈)alkylene-, —CO₂R^{a}, R^{a}C(═O)O—, R^{a}C(═O)—, —OCO₂R^{a}, R^{b}R^{c}NC(═O)O—, R^{a}OC(═O)N(R^{b})—, R^{b}R^{c}N—, R^{b}R^{c}NC(═O)—, R^{a}C(═O)N(R^{b})—, R^{b}R^{c}NC(═O)N(R^{b})—, R^{b}R^{c}NC(═S)N(R^{b})—, —OPO₃R^{a}, R^{a}OC(═S)—, R^{a}C(═S)—, —SSR^{a}, R^{a}S(═O)—, or —N═NR^{b};
each R² is independently hydrogen, halo, (C₁-C₈)alkyl, (C₃-C₈)cycloalkyl, heterocycle, heterocycle(C₁-C₈)alkylene-, aryl, aryl(C₁-C₈)alkylene-, heteroaryl, or heteroaryl(C₁-C₈)alkylene-;
alternatively, R¹ and R₂, and the atom to which they are attached is C═O, C═S or C═NR^{d},
each R⁶ is independently hydrogen, halo, —OR^{a}, —SR^{a}, (C₁-C₈)alkyl, cyano, nitro, trifluoromethyl, trifluoromethoxy, (C₁-C₈)cycloalkyl, (C₆-C₁₂)bicycloalkyl, heterocycle, heterocycle(C₁-C₈)alkylene-, aryl, aryl(C₁-C₈)alkylene-, heteroaryl, heteroaryl(C₁-C₈)alkylene-, —CO₂R^{a}, R^{a}C(═O)O—, R^{a}C(═O)—, —COO₃R^{a}, R^{b}R^{c}NC(═O)O—, R^{a}OC(═O)N(R^{b})—, R^{b}R^{c}N—, R^{b}R^{c}NC(═O)—, R^{a}C(═O)N(R^{b})—, R^{b}R^{c}NC(═O)N(R^{b})—, R^{b}R^{c}NC(═S)N(R^{b})—, —OPO₃R^{a}, R^{a}OC(═S)—, R^{a}C(═S)—, —SSR^{a}, R^{a}S(═O)—, —NNR^{b}, or two R⁶ groups and the atom to which they are attached is C═O, C═S; or two R⁶ groups with the atom or atoms to which they are attached can form a carbocyclic or heterocyclic ring including from 1-6 carbon atoms and 1, 2, 3, or 4 heteroatoms selected from —O—, S(O)₀₋₂ or amine in the ring;
each R² is independently hydrogen, (C₁-C₈)alkyl, (C₃-C₈)cycloalkyl, aryl, aryl(C₁-C₈)alkylene, heteroaryl, or heteroaryl(C₁-C₈)alkylene-;
X is —CH₂OR^{a}, —CO₂R^{a}, —CH₂OC(O)R^{a}, —C(O)NR^{b}R^{c}, —CH₂SR^{a}, —C(S)OR^{a}, —CH₂OC(S)R^{a}, —C(S)NR^{b}R^{c}, or —CH₂N(R^{b})(R^{c});
alternatively, X is a group having the formula: 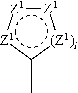
each Z¹ is —O—, S(O)₀₋₂, —C(R⁸)—, or —NR⁸—, provided that at least one Z¹ is —O—, S(O)₀₋₂, or —NR⁸—;
each R⁸ is independently hydrogen, (C₁-C₈)alkyl, (C₁-C₈)alkenyl, (C₃-C₈)cycloalkyl, (C₃-C₈)cycloalkyl(C₁-C₈)alkylene, (C₃-C₈)cycloalkenyl, (C₃-C₈)cycloalkenyl(C₁-C₈)alkylene, aryl, aryl(C₁-C₈)alkylene, heteroaryl, or heteroaryl(C₁-C₈)alkylene, wherein any of the alkyl or alkenyl groups of R⁸ are optionally interrupted by —O—, —S—, or —N(R^{a})—;
wherein any of the alkyl, cycloalkyl, heterocycle, aryl, or heteroaryl, groups of R¹, R², R⁶, R⁷ and R⁸ is optionally substituted on carbon with 1, 2, 3, or 4 substituents selected from the group consisting of halo, —OR^{a}, (C₁-C₈)alkyl, cyano, nitro, trifluoromethyl, trifluoromethoxy, (C₃-C₈)cycloalkyl, (C₀-C₁₂)bicycloalkyl, heterocycle, heterocycle(C₁-C₈)alkylene-, aryl, aryloxy, aryl(C₁-C₈)alkylene-, heteroaryl, heteroaryl(C₁-C₈)alkylene-, —CO₂R^{a}, R^{a}C(═O)O—, R^{a}C(═O)—, —OCO₂R^{a}, R^{b}R^{c}NC(═O)O—, R^{a}OC(═O)N(R^{b})—, R^{b}R^{c}NC(═O)—, R^{a}C(═O)N(R^{b})—, R^{b}R^{c}NC(═O)N(R^{b})—, R^{b}R^{c}NC(═S)N(R^{b})—, —OPO₃R^{a}, R^{a}OC(═S)—, R^{a}C(═S)—, —SSR^{a}, R^{a}S(═O)₂—, R^{b}R^{c}NS(O)ₚ—, and —N═NR^{b};
wherein any (C₁-C₈)alkyl, (C₃-C₈)cycloalkyl, (C₆-C₁₂)bicycloalkyl, (C₁-C₈)alkoxy, (C₁-C₈)alkanoyl, (C₁-C₈)alkylene, or heterocycle, is optionally partially unsaturated;
each R^{a}, R^{b} and R^{c} is independently hydrogen, (C₁-C₁₂)alkyl, (C₁-C₈)alkoxy, (C₁-C₈)alkoxy, (C₁-C₈)alkylene, (C₃-C₈)cycloalkyl, (C₃-C₈)cycloalkyl-(C₁-C₁₂)alkylene, (C₁-C₈)alkylthio, amino acid, aryl, aryl(C₁-C₈)alkylene, heterocycle, heterocycle-(C₁-C₈)alkylene, heteroaryl, or heteroaryl(C₁-C₈)alkylene;
alternatively R^{b} and R^{c}, together with the nitrogen to which they are attached, form a pyrrolidino, piperidino, morpholino, or thiomorpholino
wherein any of the alkyl, cycloalkyl, heterocycle, aryl, or heteroaryl groups of R^{a}, R^{b} and R^{c} is optionally substituted on carbon with 1 or 2 substituents selected from the group consisting of halo, —(CH₂)ₐSR^{e}, (C₁-C₈)alkyl, (CH)ₐCN, (CH₂)ₐNO₂ trifluoromethyl, trifluoromethoxy, —(CH₂)ₐCO₂R³, (CH₂)ₐNR^{e}R^{e}, and (CH₂)ₐC(O)NR^{e}R^{e};
R^{d} is hydrogen or (C₁-C₆)alkyl;
R^{e} is independently selected from H and (C₁-C₆)alkyl;
a is 0, 1, or 2;
m is 0 to 8; and
p is 0 to 2;
any mixtures thereof, any stereoisomers, or a pharmaceutically acceptable salt thereof.

23-26. (canceled)

27. The method of claim 1, wherein the A_{2A}, agonist is selected from the group consisting of: 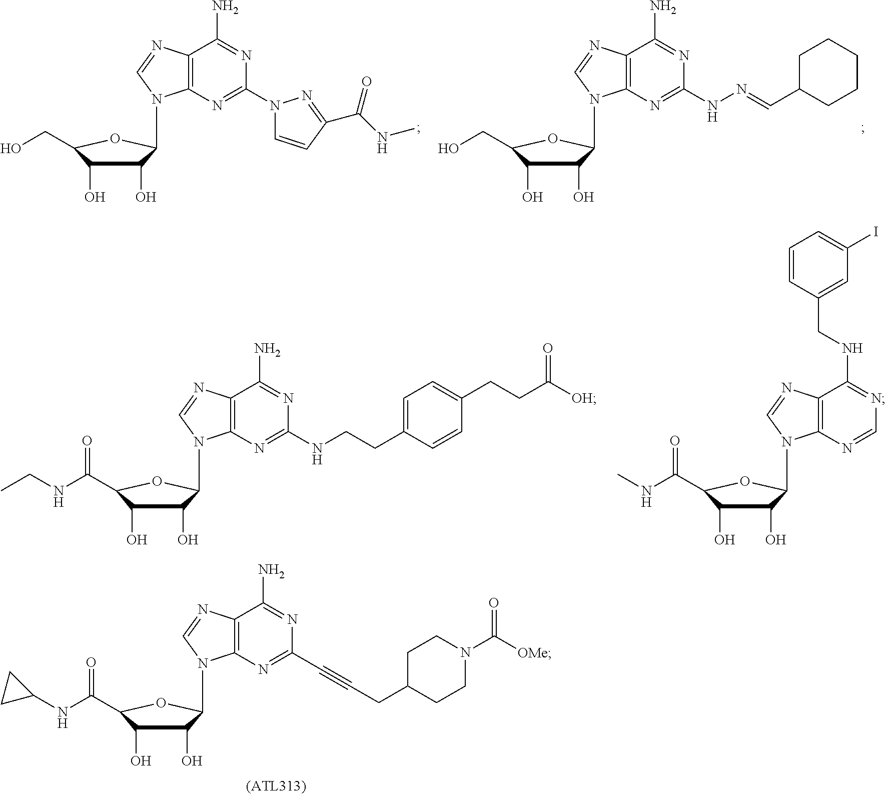
any mixtures thereof, or a pharmaceutically acceptable salt thereof:

28-30. (canceled)

31. The method of claim 1, wherein the A_{2A} agonist is administered intrathecally to the patient.

32. The method of claim 1, wherein the patient is a mammal.

33. The method of claim 32, wherein the mammal is human.
